(19) United States
(12) Patent Application Publication
(10) Pub. No.: US 2026/0263637 A1
(43) Pub. Date: Sep. 10, 2026

(54) CRISPR-RELATED METHODS AND COMPOSITIONS TARGETING PTPN2 EXPRESSION

(71) Applicant: EDITAS MEDICINE, INC., Cambridge, MA (US)

(72) Inventors: Aaron WILSON, Cambridge, MA (US); Christopher WILSON, Cambridge, MA (US)

(73) Assignee: EDITAS MEDICINE, INC., Cambridge, MA (US)

(21) Appl. No.: 19/398,688

(22) Filed: Nov. 24, 2025

Related U.S. Application Data
(63) Continuation of application No. PCT/US2024/031096, filed on May 24, 2024.
(60) Provisional application No. 63/504,651, filed on May 26, 2023.

(51) Int. Cl. A61K 48/00; C12N 5/0783; C12N 9/22; C12N 15/11; C12N 15/85

(52) U.S. Cl. CPC A61K 48/005; C12N 5/0636; C12N 9/226; C12N 15/111; C12N 15/85; C12N 2310/20; C12N 2320/33; C12N 2510/00

(57) ABSTRACT

The present disclosure relates to CRISPR-related systems and components for targeting, editing, and/or modulating expression of a PTPN2 (Protein Tyrosine Phosphatase Non-Receptor Type 2) gene. The present disclosure also relates to methods and applications thereof in connection with engineered cells including T cells or T cell precursors.

### CROSS-REFERENCE TO RELATED APPLICATIONS

This application is a continuation of International Application No. PCT/US2024/031096, filed May 24, 2024, which claims priority to U.S. Provisional Patent Application No. 63/504,651, filed on May 26, 2023, the entire contents of which are incorporated herein by reference.

### REFERENCE TO SEQUENCE LISTING SUBMITTED ELECTRONICALLY

The contents of the electronic sequence listing (084177.0324_ST26.xml; Size 212,201 bytes; and Date of Creation: Apr. 14, 2026) is herein incorporated by reference in its entirety.

### FIELD

The present disclosure is directed to CRISPR-related systems and components for targeting, editing, and/or modulating expression of a PTPN2 (Protein Tyrosine Phosphatase Non-Receptor Type 2) gene. The present disclosure is also directed to methods and applications thereof in connection with disease including, for example, engineered cells including T cells or T cell precursors.

### BACKGROUND

CRISPRs (Clustered Regularly Interspaced Short Palindromic Repeats) evolved in bacteria and archaea as an adaptive immune system to defend against viral attack. Upon exposure to a virus, short segments of viral DNA are integrated into the CRISPR locus. RNA is transcribed from a portion of the CRISPR locus that includes the viral sequence. That RNA, which contains sequence complementary to the viral genome, mediates targeting of a Cas protein to a target sequence in the viral genome. The Cas protein, in turn, cleaves and thereby silences the viral target. Naturally occurring CRISPR systems are organized evolutionarily into two classes and five types. Cas12a (also known as Cpf1) represents a Class 2, Type V CRISPR/Cas system that has been adapted for genome editing in eukaryotic cells. The introduction of site-specific double strand breaks (DSBs) into the targeted sequence allows for knocking out a gene through the formation of an indel through endogenous DNA repair mechanisms, for example non-homologous end-joining (NHEJ). The introduction of site-specific DSBs into the targeted sequence can also facilitate gene conversion or gene correction through the incorporation of an exogenous or endogenous homologous sequence with a repair template, for example homology-directed repair (HDR).

The human PTPN2 gene is located on Chromosome 18. The PTPN2 transcript (ENST00000309660.10) comprises 9 exons that encode the PTPN2 (Protein Tyrosine Phosphatase Non-Receptor Type 2) protein. PTPN2 has been shown to be involved in T cell activation and proliferation. For example, PTPN2 negatively regulates T-cell receptor (TCR) signaling by dephosphorylating lymphocyte-specific protein tyrosine kinase (LCK). PTPN2 also inhibits cytokine signaling associated with T cell activity by dephosphorylating and inactivating Janus-activated kinases (JAK-1 and JAK-3) and their target substrates (STAT-1, STAT-3, and STAT-5).

### SUMMARY

The presently disclosed subject matter relates to RNA-guided nuclease-related, e.g., CRISPR/Cas-related, methods, genome editing systems, and compositions for targeting a PTPN2 nucleic acid sequence, editing a target PTPN2 nucleic acid sequence, or modulating expression of a target PTPN2 gene. The presently disclosed subject matter also provides genome editing systems, compositions, vectors, and methods using CRISPR/Cas-related components for editing a target PTPN2 gene in cells, e.g., human T cells. Some aspects of the present disclosure provide pharmaceutical compositions, cells, cell populations, methods, strategies, and treatment modalities that are useful in the context of immunotherapeutic approaches, e.g., immunooncology therapeutic approaches. In some embodiments, the present disclosure provides modified cells e.g., T cells (or other lymphocytes) that are useful in immunotherapeutic approaches.

Provided herein are genome editing systems, RNA-guided nucleases, Cas12a (also known as Cpf1) proteins, including modified Cas12a proteins (Cas12a variants), guide RNAs, and ribonucleoprotein (RNP) complexes for modulating PTPN2 signaling. In certain embodiments, an RNP complex may include a guide RNA (gRNA) complexed to a wild-type Cas12a or modified Cas12a RNA-guided nuclease (modified Cas12a protein). In certain embodiments the modified Cas12a protein is an activity enhanced AsCas12a protein with a nuclear localization sequence (NLS). In certain embodiments, the AsCas12a protein comprises amino acid changes to increase activity and/or inactivate RNAase activity. In certain embodiments, the AsCas12a protein comprises a C-terminal linker and nuclear localization sequence. In certain embodiments, an RNP complex comprises a guide RNA (gRNA) molecule that targets a sequence of a PTPN2 gene. In certain embodiments, the RNP complex is transfected into a target cell and induces editing resulting in the formation of an indel within or near the target sequence of the PTPN2 gene. In certain embodiments, the editing results in the knockout of the PTPN2 gene.

In some embodiments the target cells provided herein are engineered cells and comprise one or more genome edits. In some embodiments, the target cells provided herein are immunocompetent cells, e.g., a T cell, a CD8+ T cell (e.g., a CD8+ naive T cell, central memory T cell, or effector memory T cell), a CD4+ T cell, an alpha/beta T cell, a gamma/delta T cell, a natural killer T cell (NKT cell), a regulatory T cell (Treg), a stem cell memory T cell, a lymphoid progenitor cell, a hematopoietic stem cell, a natural killer cell (NK cell) or a dendritic cell. In some embodiments, the target cells further comprise a chimeric antigen receptor (CAR). In some embodiments, the target cells are T cells, and the one or more edits enhance their efficacy in immunotherapeutic approaches. For example, in some embodiments, T cells are provided that comprise one or more edits that result in the loss-of-function in a gene or protein associated with inhibition of T cell function in a therapeutic context, and/or one or more modifications that effect an expression of an exogenous nucleic acid or protein associated with an enhanced T cell function in a therapeutic context. In some embodiments, the target cells provided herein comprise one or more genomic edits, e.g., indels or insertions of exogenous nucleic acid constructs resulting from cutting a genomic locus with an RNA-guided nuclease. The use of RNA-guided nuclease technology in the context of the generation of modified T cells allows for the engineering of complex alterations with enhanced characteristics relevant for clinical applications.

### BRIEF DESCRIPTION OF THE DRAWINGS

The accompanying drawings are intended to provide illustrative, and schematic rather than comprehensive, examples of certain aspects and embodiments of the present disclosure. The drawings are not intended to be limiting or binding to any particular theory or model, and are not necessarily to scale. Without limiting the foregoing, nucleic acids and polypeptides can be depicted as linear sequences, or as schematic two- or three-dimensional structures; these depictions are intended to be illustrative rather than limiting or binding to any particular model or theory regarding their structure.

FIG. 1 depicts screening results for PTPN2 targeting RNPs including different guide RNAs. Indel Fraction Window (Y-axis) is a measure of the percent of total sequencing reads that show indels (editing)+/−15 bases of the expected cut site for each given RNP. The gRNA target sequence for each RNP screened (X-axis) is listed in Table 4. RNP16 is highlighted in the box.

FIGS. 2A-2C show RNP30 concentration response data for percent editing as determined by NGS (A), and percent protein reduction determined by automated Western blot system (B and C). Results for three different donors (CEL-A, B, C) are depicted.

### DETAILED DESCRIPTION

### Definitions and Abbreviations

Unless otherwise specified, each of the following terms has the meaning associated with it in this section.

The indefinite articles “a” and “an” refer to at least one of the associated noun, and are used interchangeably with the terms “at least one” and “one or more.” For example, “a module” means at least one module, or one or more modules.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The conjunctions “or” and “and/or” are used interchangeably as non-exclusive disjunctions.

The phrase “consisting essentially of” means that the species recited are the predominant species, but that other species can be present in trace amounts or amounts that do not affect structure, function or behavior of the subject composition. For instance, a composition that consists essentially of a particular species will generally comprise 90%, 95%, 96%, or more of that species.

“Domain” is used to describe a segment of a protein or nucleic acid. Unless otherwise indicated, a domain is not required to have any specific functional property.

An “indel” is an insertion and/or deletion in a nucleic acid sequence. An indel can be the product of the repair of a DNA double strand break, such as a double strand break formed by a genome editing system of the present disclosure. An indel is most commonly formed when a break is repaired by an “error prone” repair pathway such as the NHEJ pathway described below.

“Gene conversion” refers to the alteration of a DNA sequence by incorporation of an endogenous homologous sequence (e.g. a homologous sequence within a gene array). “Gene correction” refers to the alteration of a DNA sequence by incorporation of an exogenous homologous sequence, such as an exogenous single- or double stranded donor template DNA. Gene conversion and gene correction are products of the repair of DNA double-strand breaks by HDR pathways such as those described below.

Indels, gene conversion, gene correction, and other genome editing outcomes are typically assessed by sequencing (most commonly by “next-gen” or “sequencing-by-synthesis” methods, though Sanger sequencing can still be used) and are quantified by the relative frequency of numerical changes (e.g., +1, +2 or more bases) at a site of interest among all sequencing reads. DNA samples for sequencing can be prepared by a variety of methods known in the art, and can involve the amplification of sites of interest by polymerase chain reaction (PCR), the capture of DNA ends generated by double strand breaks, as in the GUIDE-seq process described in Tsai et al. (Nat. Biotechnol. 34(5): 483 (2016), incorporated by reference herein) or by other means well known in the art. Genome editing outcomes can also be assessed by in situ hybridization methods such as the FiberComb™ system commercialized by Genomic Vision (Bagneux, France), and by any other suitable methods known in the art.

“Alt-HDR,” “alternative homology-directed repair,” or “alternative HDR” are used interchangeably to refer to the process of repairing DNA damage using a homologous nucleic acid (e.g., an endogenous homologous sequence, e.g., a sister chromatid, or an exogenous nucleic acid, e.g., a template nucleic acid). Alt-HDR is distinct from canonical HDR in that the process utilizes different pathways from canonical HDR, and can be inhibited by the canonical HDR mediators, RAD51 and BRCA2. Alt-HDR is also distinguished by the involvement of a single-stranded or nicked homologous nucleic acid template, whereas canonical HDR generally involves a double-stranded homologous template.

“Canonical HDR,” “canonical homology-directed repair” or “cHDR” refer to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA. In a normal cell, cHDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded.

Unless indicated otherwise, the term “HDR” as used herein encompasses both canonical HDR and alt-HDR.

“Non-homologous end joining” or “NHEJ” refers to ligation mediated repair and/or non-template mediated repair including canonical NHEJ (cNHEJ) and alternative NHEJ (altNHEJ), which in turn includes microhomology-mediated end joining (MMEJ), single-strand annealing (SSA), and synthesis-dependent microhomology-mediated end joining (SD-MMEJ).

“Replacement” or “replaced,” when used with reference to a modification of a molecule (e.g. a nucleic acid or protein), does not require a process limitation but merely indicates that the replacement entity is present.

“Knock-out” or “knockout” refers to an inactivating mutation in a target gene, wherein the product of the target gene comprises a loss of function.

“Gene product” refers to biochemical products resulting from the expression of the gene and includes the RNA or protein that is encoded by the gene.

“On-target site” refers to the exact genomic sequence or locus within the gene of interest for which the guide RNA was designed to target. “Off-target site” refers to a genomic sequence or locus that is not within the gene of interest and is found to be edited by the RNA guided nucleases.

“Subject” means a human or non-human animal. A human subject can be any age (e.g., an infant, child, young adult, or adult), and can suffer from a disease, or can be in need of alteration of a gene. Alternatively, the subject can be an animal, which term includes, but is not limited to, mammals, birds, fish, reptiles, amphibians, and more particularly non-human primates, rodents (such as mice, rats, hamsters, etc.), rabbits, guinea pigs, dogs, cats, and so on. In certain embodiments of this disclosure, the subject is livestock, e.g., a cow, a horse, a sheep, or a goat. In certain embodiments, the subject is poultry.

An “alpha/beta T cell” refers to a T lymphocyte that expresses an αβ T cell receptor (TCR) versus a “gamma/delta T cell” which expresses a γδ TCR.

As used herein a “therapeutically effective amount” refers to the amount of a cell and/or composition that when administered to a subject for treating a disease, is sufficient to beneficially affect such treatment for the disease.

“Treat,” “treating,” and “treatment” mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” refer to the prevention of a disease in a mammal, e.g., in a human, including (a) avoiding or precluding the disease; (b) affecting the predisposition toward the disease; or (c) preventing or delaying the onset of at least one symptom of the disease.

A “Kit” refers to any collection of two or more components that together constitute a functional unit that can be employed for a specific purpose. By way of illustration (and not limitation), one kit according to this disclosure can include a guide RNA complexed or able to complex with an RNA-guided nuclease, and accompanied by (e.g. suspended in, or suspendable in) a pharmaceutically acceptable carrier. The kit can be used to introduce the complex into, for example, a cell or a subject, for the purpose of causing a desired genomic alteration in such cell or subject. The components of a kit can be packaged together, or they can be separately packaged. Kits according to this disclosure also optionally include directions for use (DFU) that describe the use of the kit e.g., according to a method of this disclosure. The DFU can be physically packaged with the kit, or it can be made available to a user of the kit, for instance by electronic means.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional IUPAC notation is used in nucleotide sequences presented herein, as shown in Table 1, below (see also Comish-Bowden A, Nucleic Acids Res. 1985 May 10; 13(9):3021-30, incorporated by reference herein). It should be noted, however, that “T” denotes “Thymine or Uracil” in those instances where a sequence can be encoded by either DNA or RNA, for example in a gRNA, for example in a gRNA targeting domain.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

The term “variant” refers to an entity such as a polypeptide, polynucleotide or small molecule that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity.

As used herein, the term “promoter” refers to a region (i.e., a DNA sequence) of a genome that initiates the transcription of a gene.

The term “endogenous,” as used herein in the context of nucleic acids (e.g., genes, protein-encoding genomic regions, promoters), refers to a native nucleic acid or protein in its natural location, e.g., within the genome of a cell. In contrast, the term “exogenous,” as used herein in the context of nucleic acids, e.g., expression constructs, cDNAs, indels, and nucleic acid vectors, refers to nucleic acids that have artificially been introduced into the genome of a cell using, for example, gene-editing or genetic engineering techniques, e.g., CRISPR-based editing techniques.

The terms “RNA-guided nuclease” and “RNA-guided nuclease molecule” are used interchangeably herein. In some embodiments, the RNA-guided nuclease is a RNA-guided DNA endonuclease enzyme. In some embodiments, the RNA-guided nuclease is a CRISPR nuclease. Non-limiting examples of RNA-guided nucleases are listed in Table 2 below, and the methods and compositions disclosed herein can use any combination of RNA-guided nucleases disclosed herein, or known to those of ordinary skill in the art. Those of ordinary skill in the art will be aware of additional nucleases and nuclease variants suitable for use in the context of the present disclosure, and it will be understood that the present disclosure is not limited in this respect.

TABLE 2
RNA-Guided Nucleases

| | Length | | |
|---|---|---|---|
| Nuclease | (a.a.) | PAM | Reference |
| SpCas9 | 1368 | NGG | Cong et al., Science. 2013; 339(6121): 819-23 |
| SaCas9 | 1053 | NNGRRT | Ran et al., Nature. 2015; 520(7546): 186-91. |
| (KKH) | 1067 | NNNRRT | Kleinstiver et al., Nat Biotechnol. 2015; 33(12): 1293-1298 |
| SaCas9 | | | |
| AsCas12a | 1353 | TTTV | Zetsche et al., Nat Biotechnol. 2017; 35(1): 31-34. |
| (AsCpf1) | | | |
| LbCas12a | 1274 | TTTV | Zetsche et al., Cell. 2015; 163(3): 759-71. |
| (LbCpf1) | | | |
| CasX | 980 | TTC | Burstein et al., Nature. 2017; 542(7640): 237-241. |
| CasY | 1200 | TA | Burstein et al., Nature. 2017; 542(7640): 237-241. |
| Cas12hl | 870 | RTR | Yan et al., Science. 2019; 363(6422): 88-91. |
| Cas12il | 1093 | TTN | Yan et al., Science. 2019; 363(6422): 88-91. |
| Cas12cl | unknown | TG | Yan et al., Science. 2019; 363(6422): 88-91. |
| Cas12c2 | unknown | TN | Yan et al., Science. 2019; 363(6422): 88-91. |
| eSpCas9 | 1423 | NGG | Chen et al., Nature. 2017; 550(7676): 407-410. |
| Cas9-HF1 | 1367 | NGG | Chen et al., Nature. 2017; 550(7676): 407-410. |
| HypaCas9 | 1404 | NGG | Chen et al., Nature. 2017; 550(7676): 407-410. |
| dCas9-Fokl | 1623 | NGG | U.S. Pat. No. 9,322,037 |
| Sniper-Cas9 | 1389 | NGG | Lee et al., Nat Commun. 2018; 9(1): 3048. |
| xCas9 | 1786 | NGG, NG, | Wang et al., Plant Biotechnol J. 2018; pbi.13053. |
| | | GAA, GAT | |
| AaCas12b | 1129 | TTN | Teng et al. Cell Discov. 2018; 4: 63. |
| evoCas9 | 1423 | NGG | Casini et al., Nat Biotechnol. 2018; 36(3): 265-271. |
| SpCas9-NG | 1423 | NG | Nishimasu et al., Science. 2018; 361(6408): 1259-1262. |
| VRQR | 1368 | NGA | Li et al., The CRISPR Journal, 2018; 01: 01 |
| VRER | 1372 | NGCG | Kleinstiver et al., Nature. 2016; 529(7587): 490-5. |
| NmeCas9 | 1082 | NNNGATT | Amrani et al., Genome Biol. 2018; 19(1): 214. |
| CjCas9 | 984 | NNNNRYA | Kim et al., Nat Commun. 2017; 8: 14500. |
| BhCas12b | 1108 | ATTN | Strecker et al., Nat Commun. 2019 Jan 22; 10(1): 212. |
| BhCas12b | 1108 | ATTN | Strecker et al., Nat Commun. 2019 Jan 22; 10(1): 212. |
| V4 | | | |
| CasΦ | | | Pausch et al., Science 2020; 369(6501): 333-337. |

Additional suitable RNA-guided nucleases, e.g., Cas9 and Cas12 nucleases, will be apparent to the skilled artisan in view of the present disclosure, and the disclosure is not limited by the exemplary suitable nucleases provided herein. In some embodiment, a suitable nuclease is a Cas9 or Cas12a (Cpf1) nuclease. In some embodiments, the disclosure also embraces nuclease variants, e.g., Cas9 or Cas12a nuclease variants. A nuclease variant refers to a nuclease comprising an amino acid sequence characterized by one or more amino acid substitutions, deletions, or additions as compared to the wild type amino acid sequence of the nuclease. Suitable nucleases and nuclease variants may also include purification tags (e.g., polyhistidine tags) and signaling peptides, e.g., comprising or consisting of a nuclear localization signal sequence. Some non-limiting examples of suitable nucleases and nuclease variants are described in more detail elsewhere herein, and also include those described in PCT application PCT/US2019/22374, filed Mar. 14, 2019, and entitled “Systems and Methods for the Treatment of Hemoglobinopathies,” the entire contents of which are incorporated herein by reference.

In some embodiments, the RNA-guided nuclease is an Acidaminococcus sp. Cas12a (Cpf1) variant (also known as AsCas12a or AsCpf1 variant). Suitable Cas12a nuclease variants, including suitable AsCas12a variants will be known or apparent to those of ordinary skill in the art based on the present disclosure, and include, but are not limited to, the AsCas12a variants disclosed herein or otherwise known in the art. For example, in some embodiments, the RNA-guided nuclease is an Acidaminococcus sp. Cas12a RR variant (AsCas12a-RR). In another embodiment, the RNA-guided nuclease is an AsCas12a RVR variant. For example, suitable AsCas12a variants include those having an M537R substitution, an H800A substitution, and/or an F870L substitution, or any combination thereof (numbering scheme according to AsCas12a wild-type sequence). Further non-limiting examples of suitable Cas12a variants thereof are described in PCT applications: PCT/US2018/065032, filed Dec. 11, 2018, the entire contents of each of which are incorporated herein by reference.

The term “hematopoietic stem cell” as used herein, refers to CD34+ stem cells capable of giving rise to both mature myeloid and lymphoid cell types including T cells, natural killer cells, and B cells.

### Genome Editing Systems

Various genome editing systems known in the art can be used for the methods disclosed herein. Non-limiting examples of genome editing systems that can be used with the presently disclosed subject matter include, but are not limited to CRISPR systems, zinc-finger nuclease (ZFN) systems, transcription activator-like effector nuclease (TALEN) systems, meganuclease (MN) systems, MegaTAL systems, other targeted endonuclease systems, and other chimeric endonuclease systems.

In certain embodiments, the genome editing system has RNA-guided DNA editing activity. In certain embodiments, the genome editing system includes at least two components adapted from naturally occurring CRISPR systems: a guide RNA (gRNA) and an RNA-guided nuclease. These two components form a complex that is capable of associating with a specific nucleic acid sequence and optionally editing the DNA in or around that nucleic acid sequence, for instance by making one or more of a single-strand break (an SSB or nick), a double-strand break (a DSB) and/or a point mutation.

Naturally occurring CRISPR systems are organized evolutionarily into two classes and five types (Makarova et al. Nat Rev Microbiol. 2011 June; 9(6): 467-477 (Makarova), incorporated by reference herein), and while genome editing systems of the present disclosure can adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cas12a) and one or more guide RNAs (e.g., a crRNA and, optionally, a tracrRNA) that form ribonucleoprotein (RNP) complexes that associate with (i.e. target) and cleave specific loci complementary to a targeting (or spacer) sequence of the crRNA. Genome editing systems according to the present disclosure similarly target and optionally edit cellular DNA sequences, but differ significantly from CRISPR systems occurring in nature. For example, the unimolecular guide RNAs described herein do not occur in nature, and both guide RNAs and RNA-guided nucleases according to this disclosure can incorporate any number of non-naturally occurring modifications.

Genome editing systems disclosed herein can be delivered into a cell by electroporation. Other non-viral approaches can also be employed for gene editing of target cells disclosed herein. For example, a nucleic acid molecule can be introduced into cells/subjects by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Lipid nanoparticles (LNPs) or liposomes are also contemplated for delivery of nucleic acid molecules into a cell.

Genome editing systems disclosed herein can be delivered to subjects or cells using viral vectors, e.g., retroviral vectors, e.g., gamma-retroviral vectors or lentiviral vectors. Combinations of a retroviral vector and an appropriate packaging line are suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art. Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Genome editing systems can be implemented (e.g. administered or delivered to a cell or a subject) in a variety of ways, and different implementations can be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nano-particle, micelle, liposome, etc. In certain embodiments, a genome editing system is implemented as one or more nucleic acids encoding the RNA-guided nuclease and guide RNA components described above (optionally with one or more additional components). In certain embodiments, the genome editing system is implemented as one or more vectors comprising such nucleic acids, for instance a viral vector such as an adeno-associated virus. In certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or can be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (Maeder), which is incorporated by reference herein, describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e. flanking) the point mutation, and forms DSBs that flank the mutation. This, in turn, promotes deletion of the intervening sequence, including the mutation, thereby eliminating the cryptic splice site and restoring normal gene function.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111(10):E924-932, Mar. 11, 2014 (Davis) (describing Alt-HDR); Frit et al. DNA Repair 17(2014) 81-97 (Frit) (describing Alt-NHEJ); and Iyama and Wilson III, DNA Repair (Amst.) 2013-August; 12(8): 620-636 (Iyama) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a gene in or near the target sequence, without causing double-strand breaks, e.g., by causing single-strand breaks or no cleavage (i.e., no strand breaks). For example, a genome editing system can include an RNA-guided nuclease fused to a functional domain that acts on DNA, thereby modifying the target sequence or its expression. As one example, an RNA-guided nuclease can be connected to (e.g. fused to) a cytidine deaminase functional domain, and can operate by generating targeted C-to-A substitutions. An RNA-guided nuclease can also, for example, be connected to (e.g. fused to) an adenosine deaminase functional domain. Exemplary nuclease/deaminase fusions are described in Komor et al. Nature 533, 420-424 (19 May 2016) and Kantor et al., Int. J. Mol. Sci. 21(17) 6240 (2020), which are hereby incorporated by reference in its entirety. Further non-limiting examples of suitable base editors, variants thereof, and strategies for preparing RNA-guided nucleases comprising the same are described in PCT applications: PCT/US2020/016664, filed Feb. 4, 2020; PCT/US2020/018192, filed Feb. 13, 2020; PCT/US2020/049975, field Sep. 9, 2020; PCT/US2022/012054, filed Jan. 11, 2022; and PCT/US2022/078655, filed Oct. 25, 2022, the entire contents of each of which are incorporated herein by reference. Alternatively, a genome editing system can utilize a cleavage-inactivated (i.e. a “dead”) nuclease, such as a dead Cas9 (dCas9), and can operate by forming stable complexes on one or more targeted regions of cellular DNA, thereby recruiting other functional domains and/or interfering with functions involving the targeted region(s) including, without limitation, mRNA transcription, chromatin remodeling, etc.

In certain embodiments, the RNA-guided nucleases of the present disclosure can comprise a polymerase domain (e.g., a reverse transcriptase domain). In certain embodiments, the RNA-guided nuclease may use a gRNA with a primer binding sequence and/or a template for the polymerase domain.

In certain embodiments, the RNA-guided nuclease may be a prime editor (PE), where the PE is an RNA-guided nuclease with nickase activity that is fused to a reverse transcriptase domain. In certain embodiments, the PE may use a prime editing gRNA (pegRNA), where the pegRNA is a gRNA with a primer binding sequence (PBS) and a donor template, e.g., added at one of the termini, e.g., the 3′ end. In certain embodiments, a PE:pegRNA complex binds to the target DNA, and the nickase domain of the prime editor nicks only one strand, generating a flap. The PBS, located on the pegRNA, binds to the DNA flap and the edited RNA sequence is reverse transcribed using the reverse transcriptase domain of the prime editor. The edited strand is incorporated into the DNA at the end of the nicked flap, and the target DNA is repaired with the new reverse transcribed DNA. The original DNA segment is removed by a cellular endonuclease. Additional methods employing RNA-guided nucleases and polymerases for template mediated gene editing are described in PCT publications: WO 2020/191233, WO 2020/191248, WO 2021226558, WO2023283246, WO 2023/235501, and WO 2023/076898, each of which are incorporated by reference for all purposes herein.

### Guide RNA (QRNA) Molecules

The terms “guide molecule,” “guide RNA” and “gRNA” refer to any nucleic acid that promotes the specific association (or “targeting”) of an RNA-guided nuclease such as a Cas9 or a Cas12a (Cpf1) to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (Briner), which is incorporated by reference), and in Cotta-Ramusino. The guide molecule can be an RNA molecule. The guide molecule can also comprise one or more nucleotides other than RNA nucleotides, for example, the guide molecule can be a DNA/RNA hybrid molecule, and/or the guide molecule can comprise one or more modified nucleotides (including, but not limited to, one or more modified DNA or RNA nucleotides).

In bacteria and archaea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. This duplex can facilitate the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). (Mali et al. Science. 2013 Feb. 15; 339(6121): 823-826 (“Mali”); Jiang et al. Nat Biotechnol. 2013 March; 31(3): 233-239 (“Jiang”); and Jinek et al., 2012 Science August 17; 337(6096): 816-821 (“Jinek”), all of which are incorporated by reference herein.)

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832, (“Hsu”), incorporated by reference herein), “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cas12a gRNA.

In addition to the targeting domains, gRNAs typically (but not necessarily, as discussed below) include a plurality of domains that can influence the formation or activity of gRNA/Cas complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. (Nishimasu et al., Cell 156, 935-949, Feb. 27, 2014 (Nishimasu 2014) and Nishimasu et al., Cell 162, 1113-1126, Aug. 27, 2015 (Nishimasu 2015), both incorporated by reference herein). It should be noted that the first and/or second complementarity domains can contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or can in the future be) discovered or invented which utilize gRNAs that differ in some ways from those described to this point. For instance, Cas12a (also known as Cpf1; “CRISPR from Prevotella and Franciscella 1”) is a RNA-guided nuclease that does not require a tracrRNA to function. (Zetsche et al., 2015, Cell 163, 759-771 Oct. 22, 2015 (Zetsche I), incorporated by reference herein). A gRNA for use in a Cas12a genome editing system generally includes a targeting domain and a complementarity domain (alternately referred to as a “handle”). It should also be noted that, in gRNAs for use with Cas12a, the targeting domain is usually present at or near the 3′ end, rather than the 5′ end as described above in connection with Cas9 gRNAs (the handle is at or near the 5′ end of a Cas12a gRNA).

Those of skill in the art will appreciate that, although structural differences can exist between gRNAs from different prokaryotic species, or between Cas12a and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs can be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular RNA-guided nuclease, e.g., a particular species of Cas9 or Cas12a. By way of illustration, the term gRNA can, in certain embodiments, include a gRNA for use with any RNA-guided nuclease occurring in a Class 2 CRISPR system, such as a type II or type V CRISPR system, or an RNA-guided nuclease derived or adapted therefrom.

In some embodiments, the guide RNA used comprises a modification as compared to the standard gRNA scaffold. Such modifications may comprise, for example, chemical modifications of a part of the gRNA, e.g., of a nucleobase or backbone moiety. In some embodiments, such a modification may also include the presence of a DNA nucleotide within the gRNA, e.g., within or outside of the targeting domain. In some embodiments, the modification may include an extension of the gRNA scaffold, e.g., by addition of 1-100 nucleotides, including RNA and/or DNA nucleotides at the 3′ or the 5′ terminus of the guide RNA, e.g., at the terminus distal to the targeting domain.

In certain embodiments, a gRNA complexed to an unmodified or modified Cas12a protein may be modified to increase the editing efficiency of a target nucleic acid. In certain embodiments, the modified gRNA may comprise one or more modifications including a phosphorothioate (PS2) linkage modification, a 2′-O-methyl modification, one or more or a stretch of deoxyribonucleic acid (DNA) base (also referred herein as a “DNA extension”), or combinations thereof.

In some embodiments, a gRNA disclosed herein includes one or more or a stretch of deoxyribonucleic acid (DNA) bases, also referred to herein as a “DNA extension.” In some embodiments, a gRNA disclosed herein includes a DNA extension at the 5′ end of the gRNA, the 3′ end of the gRNA, or a combination thereof. In certain embodiments, the DNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DNA bases long. For example, in certain embodiments, the DNA extension may be 1, 2, 3, 4, 5, 10, 15, 20, or 25 DNA bases long. In certain embodiments, the DNA extension may include one or more DNA bases selected from adenine (A), guanine (G), cytosine (C), or thymine (T). In certain embodiments, the DNA extension includes the same DNA bases. For example, the DNA extension may include a stretch of adenine (A) bases. In certain embodiments, the DNA extension may include a stretch of thymine (T) bases. In certain embodiments, the DNA extension includes a combination of different DNA bases. In certain embodiments, a DNA extension may comprise or consist of a sequence set forth in Table 3. In certain embodiments, a gRNA disclosed herein includes a DNA extension as well as one or more phosphorothioate linkage modifications, one or more phosphorodithioate (PS2) linkage modifications, one or more 2′-O-methyl modifications, or combinations thereof. In certain embodiments, the one or more modifications may be at the 5′ end of the gRNA, at the 3′ end of the gRNA, or combinations thereof. In certain embodiments, a gRNA including a DNA extension may comprise a sequence set forth in Table 3 that includes a DNA extension. Without wishing to be bound by theory, it is contemplated that any DNA extension may be used herein, so long as it does not hybridize to the target nucleic acid being targeted by the gRNA. In some embodiments the DNA extension additionally exhibits an increase in editing efficiency, e.g., via changes to gRNA stability, uptake, and/or activity, at the target nucleic acid site relative to a gRNA which does not comprise such a DNA extension.

In some embodiments, a gRNA disclosed herein includes one or more or a stretch of ribonucleic acid (RNA) bases, also referred to herein as an “RNA extension.” In some embodiments, a gRNA disclosed herein includes an RNA extension at the 5′ end of the gRNA, the 3′ end of the gRNA, or a combination thereof. In certain embodiments, the RNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 RNA bases long. For example, in certain embodiments, the RNA extension may be 1, 2, 3, 4, 5, 10, 15, 20, or 25 RNA bases long. In certain embodiments, the RNA extension may include one or more RNA bases selected from adenine (rA), guanine (rG), cytosine (rC), or uracil (rU), in which the “r” represents RNA, 2′-hydroxy. In certain embodiments, the RNA extension includes the same RNA bases. For example, the RNA extension may include a stretch of adenine (rA) bases. In certain embodiments, the RNA extension includes a combination of different RNA bases. In certain embodiments, an RNA extension may comprise or consist of a sequence set forth in Table 3. In certain embodiments, a gRNA disclosed herein includes an RNA extension as well as one or more phosphorothioate linkage modifications, one or more phosphorodithioate (PS2) linkage modifications, one or more 2′-O-methyl modifications, or combinations thereof. In certain embodiments, the one or more modifications may be at the 5′ end of the gRNA, at the 3′ end of the gRNA, or combinations thereof. In certain embodiments, a gRNA including a RNA extension may comprise a sequence set forth in Table 3 that includes an RNA extension. gRNAs including an RNA extension at the 5′ end of the gRNA may comprise a sequence disclosed herein. gRNAs including an RNA extension at the 3′ end of the gRNA may comprise a sequence disclosed herein.

It is contemplated that gRNAs disclosed herein may also include an RNA extension and a DNA extension. In certain embodiments, the RNA extension and DNA extension may both be at the 5′ end of the gRNA, the 3′ end of the gRNA, or a combination thereof. In certain embodiments, the RNA extension is at the 5′ end of the gRNA and the DNA extension is at the 3′ end of the gRNA. In certain embodiments, the RNA extension is at the 3′ end of the gRNA and the DNA extension is at the 5′ end of the gRNA.

In some embodiments, a gRNA which includes a modification, e.g., a DNA extension at the 5′ end, is complexed with a RNA-guided nuclease, e.g., an AsCas12a nuclease, to form an RNP, which is then employed to edit a target cell, e.g., a T cell. Exemplary suitable 5′ extensions for guide RNAs, e.g., Cas12a guide RNAs, are provided in the table below:

TABLE 3
gRNA 5′ Extensions

| 5′ extension | | |
|---|---|---|
| Sequence ID NO: | 5′ extension sequence | 5′ modification |
| 1 | rCrUrUrUrU | +5 RNA |
| 2 | rArArGrArCrCrUrUrUrU | +10 RNA |
| 3 | rArUrGrUrGrUrUrUrUrUrGrUrCrArArArArGrArCrCrU | +25 RNA |
| | rUrUrU | |
| 4 | ArGrGrCrCrArGrCrUrUrGrCrCrGrGrUrUrUrUrUrUrA | +60 RNA |
| | rGrUrCrGrUrGrCrUrGrCrUrUrCrArUrGrUrGrUrUrUrU | |
| | rUrGrUrCrArArArArGrArCrCrUrUrUrU | |
| 5 | CTTTT | +5 DNA |
| 6 | AAGACCTTTT | +10 DNA |
| 7 | ATGTGTTTTTGTCAAAAGACCTTTT | +25 DNA |
| 8 | AGGCCAGCTTGCCGGTTTTTTAGTCGTGCTGCTT | +60 DNA |
| | CATGTG | |
| 9 | TTTTTGTCAAAAGACCTTTT | +20 DNA |
| 10 | GCTTCATGTGTTTTTGTCAAAAGACCTTTT | +30 DNA |
| 11 | GCCGGTTTTTTAGTCGTGCTGCTTCATGTGTTTTT | +50 DNA |
| | GTCAAAAGACCTTTT | |
| 12 | TAGTCGTGCTGCTTCATGTGTTTTTGTCAAAAGA | +40 DNA |
| | CCTTTT | |
| 13 | C*C*GAAGTTTTCTTCGGTTTT | +20 DNA + 2xPS |
| 14 | T*T*TTTCCGAAGTTTTCTTCGGTTTT | +25 DNA + 2xPS |
| 15 | A*A* CGCTTTTTCCGAAGTTTTCTTCGGTTTT | +30 DNA + 2xPS |
| 16 | G*C*GTTGTTTTCAACGCTTTTTCCGAAGTTTTCT | +41 DNA + 2xPS |
| | TCGGTTTT | |
| 17 | G*G*CTTCTTTTGAAGCCTTTTTGCGTTGTTTTCA | +62 DNA + 2xPS |
| | ACGCTTTTTCCGAAGTTTTCTTCGGTTTT | |
| 18 | A*T*GTGTTTTTGTCAAAAGACCTTTT | +25 DNA + 2xPS |
| 19 | AAAAAAAAAAAAAAAAAAAAAAAAA | +25 A |
| 20 | TTTTTTTTTTTTTTTTTTTTTTTTT | +25 T |
| 21 | mA*mU*rGrUrGrUrUrUrUrUrGrUrCrArArArArGrArC | +25 RNA + 2xPS |
| | rCrUrUrUrU | |
| 22 | mA*mA*rArArArArArArArArArArArArArArArArArA | Poly A RNA + |
| | rArArArArA | 2xPS |
| 23 | mU*mU*rUrUrUrUrUrUrUrUrUrUrUrUrUrUrUrUrUrU | PolyU RNA + |
| | rUrUrUrUrU | 2xPS |

All bases are in upper case
Lowercase “r” represents RNA, 2′-hydroxy; bases not modified by an “r” are DNA
All bases are linked via standard phosphodiester bonds except as noted:
“*” represents phosphorothioate modification
“PS” represents phosphorothioate modification

Additional suitable gRNA modifications will be apparent to those of ordinary skill in the art based on the present disclosure. Suitable gRNA modifications include, for example, those described in PCT application PCT/US2018/054027, filed on Oct. 2, 2018, and entitled “MODIFIED CPF1 GUIDE RNA;” in PCT application PCT/US2015/000143, filed on Dec. 3, 2015, and entitled “GUIDE RNA WITH CHEMICAL MODIFICATIONS;” in PCT application PCT/US2016/026028, filed Apr. 5, 2016, and entitled “CHEMICALLY MODIFIED GUIDE RNAS FOR CRISPR/CAS-MEDIATED GENE REGULATION;” and in PCT application PCT/US2016/053344, filed on Sep. 23, 2016, and entitled “NUCLEASE-MEDIATED GENOME EDITING OF PRIMARY CELLS AND ENRICHMENT THEREOF;” the entire contents of each of which are incorporated herein by reference.

gRNA Design

Methods for selection and validation of target sequences as well as off-target analyses have been described previously, e.g., in Mali; Hsu; Fu et al., 2014 Nat biotechnol 32(3): 279-84, Heigwer et al., 2014 Nat methods 11(2):122-3; Bae et al. (2014) Bioinformatics 30(10): 1473-5; and Xiao A et al. (2014) Bioinformatics 30(8): 1180-1182. Each of these references is incorporated by reference herein. In certain non-limiting embodiments, gRNA design can involve the use of a software tool to optimize the choice of potential target sequences corresponding to a user's target sequence, e.g., to minimize total off-target activity across the genome. These and other guide selection methods are described in detail in Maeder and Cotta-Ramusino.

In certain embodiments, one or more or all of the nucleotides in a gRNA are modified. Strategies for modifying a gRNA are described in WO2019/152519, published Aug. 8, 2019, the entire contents of which are expressly incorporated herein by reference.

Non-limiting examples of guide RNAs suitable for certain embodiments embraced by the present disclosure are provided herein, for example, in the Tables below. Those of ordinary skill in the art will be able to envision suitable guide RNA sequences for a specific nuclease, e.g., a Cas9 or Cas12a nuclease, from the disclosure of the targeting domain sequence, either as a DNA or RNA sequence. For example, a guide RNA comprising a targeting sequence consisting of RNA nucleotides would include the RNA sequence corresponding to the targeting domain sequence provided as a DNA sequence, and thus contain uracil instead of thymidine nucleotides. For example, a guide RNA comprising a targeting domain sequence consisting of RNA nucleotides, and described by the DNA sequence GAGTGGTGAAACCAGAACAAT (SEQ ID NO: 39) would have a targeting domain of the corresponding RNA sequence rGrArGrUrGrGrUrGrArArArCrCrArGrArArCrArArU (SEQ ID NO: 68). As will be apparent to the skilled artisan, such a targeting sequence would be linked to a suitable guide RNA scaffold, e.g., a crRNA scaffold sequence or a chimeric crRNA/tracrRNA scaffold sequence. Suitable gRNA scaffold sequences are known to those of ordinary skill in the art. For AsCas12a, for example, a suitable scaffold sequence comprises the sequence rUrArArUrUrUrCrUrArCrUrCrUrUrGrUrArGrArU (SEQ ID NO: 111), added to the 5′-terminus of the targeting domain. In the example above, this would result in a Cas12a guide RNA of the sequence rUrArArUrUrUrCrUrArCrUrCrUrUrGrUrArGrArUrGrArGrUrGrGrUrGrArArArCrCrAr GrArArCrArArU (SEQ ID NO: 97). Those of skill in the art would further understand how to modify such a guide RNA. For example, adding a 25-mer DNA extension (SEQ ID NO: 7) would result, for example, in a guide RNA of the sequence ATGTGTTTTTGTCAAAAGACCTTTTrUrArArUrUrUrCrUrArCrUrCrUrUrGrUrArGrArUrGrA rGrUrGrGrUrGrArArArCrCrArGrArArCrArArU (SEQ ID NO: 112). It will be understood that the exemplary targeting sequences provided herein are not limiting, and additional suitable sequences, e.g., variants of the specific sequences disclosed herein, will be apparent to the skilled artisan based on the present disclosure in view of the general knowledge in the art.

In some embodiments, the gRNA for use in the disclosure is a gRNA targeting PTPN2 (PTPN2 gRNA). In some embodiments, the target sequence of a PTPN2 gene comprises or consists of a nucleotide sequence that is a least 10, at least 16, at least 17, at least 18, at least 20, or at least 21 nucleotides in length. In certain embodiments, the target sequence or target locus of PTPN2 gene has at least 50%, 60%, 70%, 75%, 80%, 85%, 90%, or 95% sequence identity to a nucleotide sequence set forth in SEQ ID NOs: 24-52. In some embodiments, the target sequence or target locus of a PTPN2 gene has 1, 2, 3, 4, 5, 6, 7, 8, 9, or 10 mutations relative to a nucleotide sequence set forth in SEQ ID NOs: 24-52. In some embodiments, the target sequence or target locus of a PTPN2 gene has less than 2, 3, 4, 5, 6, 7, 8, 9, or 10 mutations relative to a nucleotide sequence set forth in SEQ ID NOs: 24-52. In some embodiments, the target sequence of a PTPN2 gene comprises or consists of a nucleotide sequence set forth in SEQ ID NOs: 24-52. In some embodiments, the target locus of a PTPN2 gene comprises a nucleotide sequence set forth in SEQ ID NOs: 24-52.

In certain embodiments, the target sequence or target locus of a PTPN2 gene is in exon 6 of PTPN2. In certain embodiments, the target sequence of a PTPN2 gene comprises or consists of a nucleotide sequence that is a least 10, at least 16, at least 17, at least 18, at least 20, or at least 21 nucleotides in length. In certain embodiments, the target sequence of PTPN2 gene has at least 50%, 60%, 70%, 75%, 80%, 85%, 90%, or 95% sequence identity to a nucleotide sequence set forth in SEQ ID NO: 131. SEQ ID NO: 131 is set forth below.

[SEQ ID NO: 131]
ccttgattttcactttttagAGTGGTGAAACCAGAACAATATCTCACTTT
CATTATACTACCTGGCCAGATTTTGGAGTCCCTGAATCACCAGCTTCATT
TCTCAATTTCTTGTTTAAAGTGAGAGAATCTGGCTCCTTGAACCCTGACC
ATGGGCCTGCGGTGATCCACTGTAGTGCAGGCATTGGGCGCTCTGGCACC
TTCTCTCTGGTAGACACTTGTCTTGTTTTGgtaagtgcttcttacgatct

In certain embodiments, the target sequence of a PTPN2 gene comprises or consists of the nucleotides set forth in SEQ ID NO: 29, SEQ ID NO: 36, SEQ ID NO: 39, or SEQ ID NO: 51. In certain embodiments, the target sequence of a PTPN2 gene comprises or consists of the nucleotide sequence set forth in SEQ ID NO: 39.

In certain embodiments, the targeting domain of the gRNA can be complementary to either strand of a target sequence or locus of a PTPN2 gene. In some embodiments, the targeting domain of the gRNA molecule has at least 50%, 60%, 70%, 75%, 80%, 85%, 90%, or 95% sequence identity to a nucleotide sequence set forth in SEQ ID NOs: 53-81. In some embodiments, the targeting domain of the gRNA molecule has 1, 2, 3, 4, 5, 6, 7, 8, 9, or 10 mutations relative to a nucleotide sequence set forth in SEQ ID NOs: 53-81. In some embodiments, the targeting domain of the gRNA molecule has less than 2, 3, 4, 5, 6, 7, 8, 9, or 10 mutations relative to a nucleotide sequence set forth in SEQ ID NOs: 53-81. In some embodiments, the targeting domain of the gRNA molecule comprises or consists of a nucleotide sequence set forth in SEQ ID NOs: 53-81. In certain embodiments, the targeting domain of the gRNA targeting a PTPN2 gene is SEQ ID NO: 68.

In some embodiments, the gRNA molecule targeting a PTPN2 gene has at least 50%, 60%, 70%, 75%, 80%, 85%, 90%, or 95% sequence identity to a nucleotide sequence set forth in SEQ ID NOs: 82-110. In some embodiments, the gRNA molecule targeting a PTPN2 gene has 1, 2, 3, 4, 5, 6, 7, 8, 9, or 10 mutations relative to a nucleotide sequence set forth in SEQ ID NOs: 82-110. In some embodiments, the gRNA molecule targeting a PTPN2 gene has less than 2, 3, 4, 5, 6, 7, 8, 9, or 10 mutations relative to a nucleotide sequence set forth in SEQ ID NOs: 82-110. In certain embodiments, the gRNA molecule targeting a PTPN2 gene comprises or consists of a nucleotide sequence set forth in SEQ ID NOs: 82-110. In certain embodiments, the gRNA molecule targeting a PTPN2 gene comprises or consists of the sequence set forth in SEQ ID NO: 97. An exemplary PTPN2 gene target sequence, gRNA targeting domain, scaffold sequence, and DNA extension are set forth in Table 7.

TABLE 4
PTPN2 Target Sequences

| SEQ ID | | gRNA Target | Target |
|---|---|---|---|
| NO. | RNP ID | Sequence (DNA) | Site ID |
| 24 | RNP1 | TAAGGAAGACTTATCTCCTGC | RefSeq1 |
| 25 | RNP2 | TGCCTTTCTTGCAGATGGAAA | RefSeq2 |
| 26 | RNP3 | GGTTAAATGTGCACAGTACTG | RefSeq3 |
| 27 | RNP4 | TTATTTTAAGGAAATTCGAAA | RefSeq4 |
| 28 | RNP5 | CCTGTATACTAGAATCTCCCT | RefSeq5 |
| 29 | RNP6 | TAGAGTGGTGAAACCAGAACA | RefSeq6 |
| 30 | RNP7 | CAGATGATCACAGTCGTGTTA | RefSeq7 |
| 31 | RNP8 | TCTTCCCTACCTAGAAACGAT | RefSeq8 |
| 32 | RNP9 | TTGCCTTTCTTGCAGATGGAA | RefSeq9 |
| 33 | RNP10 | TAAATTTAGGTTAAATGTGCA | RefSeq10 |
| 34 | RNP11 | GTTATTTTAAGGAAATTCGAA | RefSeq11 |
| 35 | RNP12 | TTGCAGATGGAAAAAGGAGAT | RefSeq12 |
| 36 | RNP13 | ACTTTTTAGAGTGGTGAAACC | RefSeq13 |
| 37 | RNP14 | TCATTCTCTATTTTCAGATGA | RefSeq14 |
| 38 | RNP15 | ATTTCTCTTCCCTACCTAGAA | RefSeq15 |
| 39 | RNP16 | GAGTGGTGAAACCAGAACAAT | RefSeq16 |
| 40 | RNP17 | GTTGACATAGAAGAGGCACAA | RefSeq17 |
| 41 | RNP18 | TGTTATTTTAAGGAAATTCGA | RefSeq18 |
| 42 | RNP19 | AGAGGAAAGTCCTGTACATCG | RefSeq19 |
| 43 | RNP20 | CCTTTCTTGCAGATGGAAAAA | RefSeq20 |
| 44 | RNP21 | CACTTTTTAGAGTGGTGAAAC | RefSeq21 |
| 45 | RNP22 | AGGAAATTCGAAATGAGTCCC | RefSeq22 |
| 46 | RNP23 | GAGGAAAGTCCTGTACATCGG | RefSeq23 |
| 47 | RNP24 | GCCTTTCTTGCAGATGGAAAA | RefSeq24 |
| 48 | RNP25 | GGGCAAGAAAGGTCTTACATT | RefSeq25 |
| 49 | RNP26 | AAGGAAATTCGAAATGAGTCC | RefSeq26 |
| 50 | RNP27 | AAACCTGTATGAAGTTTACCT | RefSeq27 |
| 51 | RNP28 | AGAGTGGTGAAACCAGAACAA | RefSeq28 |
| 52 | RNP29 | AGATGATCACAGTCGTGTTAA | RefSeq29 |

TABLE 5
PTPN2 Guide Sequences

| SEQ ID | | Target |
|---|---|---|
| NO. | gRNA Target Sequence (RNA) | Site ID |
| 53 | rUrArArGrGrArArGrArCrUrUrArUrCrUrCrCrUrGrC | RefSeq1 |
| 54 | rUrGrCrCrUrUrUrCrUrUrGrCrArGrArUrGrGrArArA | RefSeq2 |
| 55 | rGrGrUrUrArArArUrGrUrGrCrArCrArGrUrArCrUrG | RefSeq3 |
| 56 | rUrUrArUrUrUrUrArArGrGrArArArUrUrCrGrArArA | RefSeq4 |
| 57 | rCrCrUrGrUrArUrArCrUrArGrArArUrCrUrCrCrCrU | RefSeq5 |
| 58 | rUrArGrArGrUrGrGrUrGrArArArCrCrArGrArArCrA | RefSeq6 |
| 59 | rCrArGrArUrGrArUrCrArCrArGrUrCrGrUrGrUrUrA | RefSeq7 |
| 60 | rUrCrUrUrCrCrCrUrArCrCrUrArGrArArArCrGrArU | RefSeq8 |
| 61 | rUrUrGrCrCrUrUrUrCrUrUrGrCrArGrArUrGrGrArA | RefSeq9 |
| 62 | rUrArArArUrUrUrArGrGrUrUrArArArUrGrUrGrCrA | RefSeq10 |
| 63 | rGrUrUrArUrUrUrUrArArGrGrArArArUrUrCrGrArA | RefSeq11 |
| 64 | rUrUrGrCrArGrArUrGrGrArArArArArGrGrArGrArU | RefSeq12 |
| 65 | rArCrUrUrUrUrUrArGrArGrUrGrGrUrGrArArArCrC | RefSeq13 |
| 66 | rUrCrArUrUrCrUrCrUrArUrUrUrUrCrArGrArUrGrA | RefSeq14 |
| 67 | rArUrUrUrCrUrCrUrUrCrCrCrUrArCrCrUrArGrArA | RefSeq15 |
| 68 | rGrArGrUrGrGrUrGrArArArCrCrArGrArArCrArArU | RefSeq16 |
| 69 | rGrUrUrGrArCrArUrArGrArArGrArGrGrCrArCrArA | RefSeq17 |
| 70 | rUrGrUrUrArUrUrUrUrArArGrGrArArArUrUrCrGrA | RefSeq18 |
| 71 | rArGrArGrGrArArArGrUrCrCrUrGrUrArCrArUrCrG | RefSeq19 |
| 72 | rCrCrUrUrUrCrUrUrGrCrArGrArUrGrGrArArArArA | RefSeq20 |
| 73 | rCrArCrUrUrUrUrUrArGrArGrUrGrGrUrGrArArArC | RefSeq21 |
| 74 | rArGrGrArArArUrUrCrGrArArArUrGrArGrUrCrCrC | RefSeq22 |
| 75 | rGrArGrGrArArArGrUrCrCrUrGrUrArCrArUrCrGrG | RefSeq23 |
| 76 | rGrCrCrUrUrUrCrUrUrGrCrArGrArUrGrGrArArArA | RefSeq24 |
| 77 | rGrGrGrCrArArGrArArArGrGrUrCrUrUrArCrArUrU | RefSeq25 |
| 78 | rArArGrGrArArArUrUrCrGrArArArUrGrArGrUrCrC | RefSeq26 |
| 79 | rArArArCrCrUrGrUrArUrGrArArGrUrUrUrArCrCrU | RefSeq27 |
| 80 | rArGrArGrUrGrGrUrGrArArArCrCrArGrArArCrArA | RefSeq28 |
| 81 | rArGrArUrGrArUrCrArCrArGrUrCrGrUrGrUrUrArA | RefSeq29 |

TABLE 6
PTPN2 (crRNA oligonucleotides)

| SEQ ID | RNP | | Target |
|---|---|---|---|
| NO. | ID | crRNA | Site ID |
| 82 | RNP1 | rUrArArUrUrUrCrUrArCrUrCrUrUrGrUrArGrArUrUrArAr | RefSeq1 |
| | | GrGrArArGrArCrUrUrArUrCrUrCrCrUrGrC | |
| 83 | RNP2 | rUrArArUrUrUrCrUrArCrUrCrUrUrGrUrArGrArUrUrGrCr | RefSeq2 |
| | | CrUrUrUrCrUrUrGrCrArGrArUrGrGrArArA | |
| 84 | RNP3 | rUrArArUrUrUrCrUrArCrUrCrUrUrGrUrArGrArUrGrGrUr | RefSeq3 |
| | | UrArArArUrGrUrGrCrArCrArGrUrArCrUrG | |
| 85 | RNP4 | rUrArArUrUrUrCrUrArCrUrCrUrUrGrUrArGrArUrUrUrAr | RefSeq4 |
| | | UrUrUrUrArArGrGrArArArUrUrCrGrArArA | |
| 86 | RNP5 | rUrArArUrUrUrCrUrArCrUrCrUrUrGrUrArGrArUrCrCrUr | RefSeq5 |
| | | GrUrArUrArCrUrArGrArArUrCrUrCrCrCrU | |
| 87 | RNP6 | rUrArArUrUrUrCrUrArCrUrCrUrUrGrUrArGrArUrUrArGr | RefSeq6 |
| | | ArGrUrGrGrUrGrArArArCrCrArGrArArCrA | |
| 88 | RNP7 | rUrArArUrUrUrCrUrArCrUrCrUrUrGrUrArGrArUrCrArGr | RefSeq7 |
| | | ArUrGrArUrCrArCrArGrUrCrGrUrGrUrUrA | |
| 89 | RNP8 | rUrArArUrUrUrCrUrArCrUrCrUrUrGrUrArGrArUrUrCrUr | RefSeq8 |
| | | UrCrCrCrUrArCrCrUrArGrArArArCrGrArU | |
| 90 | RNP9 | rUrArArUrUrUrCrUrArCrUrCrUrUrGrUrArGrArUrUrUrGr | RefSeq9 |
| | | CrCrUrUrUrCrUrUrGrCrArGrArUrGrGrArA | |
| 91 | RNP10 | rUrArArUrUrUrCrUrArCrUrCrUrUrGrUrArGrArUrUrArAr | RefSeq10 |
| | | ArUrUrUrArGrGrUrUrArArArUrGrUrGrCrA | |
| 92 | RNP11 | rUrArArUrUrUrCrUrArCrUrCrUrUrGrUrArGrArUrGrUrUr | RefSeq11 |
| | | ArUrUrUrUrArArGrGrArArArUrUrCrGrArA | |
| 93 | RNP12 | rUrArArUrUrUrCrUrArCrUrCrUrUrGrUrArGrArUrUrUrGr | RefSeq12 |
| | | CrArGrArUrGrGrArArArArArGrGrArGrArU | |
| 94 | RNP13 | rUrArArUrUrUrCrUrArCrUrCrUrUrGrUrArGrArUrArCrUr | RefSeq13 |
| | | UrUrUrUrArGrArGrUrGrGrUrGrArArArCrC | |
| 95 | RNP14 | rUrArArUrUrUrCrUrArCrUrCrUrUrGrUrArGrArUrUrCrAr | RefSeq14 |
| | | UrUrCrUrCrUrArUrUrUrUrCrArGrArUrGrA | |
| 96 | RNP15 | rUrArArUrUrUrCrUrArCrUrCrUrUrGrUrArGrArUrArUrUr | RefSeq15 |
| | | UrCrUrCrUrUrCrCrCrUrArCrCrUrArGrArA | |
| 97 | RNP16 | rUrArArUrUrUrCrUrArCrUrCrUrUrGrUrArGrArUrGrArGr | RefSeq16 |
| | | UrGrGrUrGrArArArCrCrArGrArArCrArArU | |
| 98 | RNP17 | rUrArArUrUrUrCrUrArCrUrCrUrUrGrUrArGrArUrGrUrUr | RefSeq17 |
| | | GrArCrArUrArGrArArGrArGrGrCrArCrArA | |
| 99 | RNP18 | rUrArArUrUrUrCrUrArCrUrCrUrUrGrUrArGrArUrUrGrUr | RefSeq18 |
| | | UrArUrUrUrUrArArGrGrArArArUrUrCrGrA | |
| 100 | RNP19 | rUrArArUrUrUrCrUrArCrUrCrUrUrGrUrArGrArUrArGrAr | RefSeq19 |
| | | GrGrArArArGrUrCrCrUrGrUrArCrArUrCrG | |
| 101 | RNP20 | rUrArArUrUrUrCrUrArCrUrCrUrUrGrUrArGrArUrCrCrUr | RefSeq20 |
| | | UrUrCrUrUrGrCrArGrArUrGrGrArArArArA | |
| 102 | RNP21 | rUrArArUrUrUrCrUrArCrUrCrUrUrGrUrArGrArUrCrArCr | RefSeq21 |
| | | UrUrUrUrUrArGrArGrUrGrGrUrGrArArArC | |
| 103 | RNP22 | rUrArArUrUrUrCrUrArCrUrCrUrUrGrUrArGrArUrArGrGr | RefSeq22 |
| | | ArArArUrUrCrGrArArArUrGrArGrUrCrCrC | |
| 104 | RNP23 | rUrArArUrUrUrCrUrArCrUrCrUrUrGrUrArGrArUrGrArGr | RefSeq23 |
| | | GrArArArGrUrCrCrUrGrUrArCrArUrCrGrG | |
| 105 | RNP24 | rUrArArUrUrUrCrUrArCrUrCrUrUrGrUrArGrArUrGrCrCr | RefSeq24 |
| | | UrUrUrCrUrUrGrCrArGrArUrGrGrArArArA | |
| 106 | RNP25 | rUrArArUrUrUrCrUrArCrUrCrUrUrGrUrArGrArUrGrGrGr | RefSeq25 |
| | | CrArArGrArArArGrGrUrCrUrUrArCrArUrU | |
| 107 | RNP26 | rUrArArUrUrUrCrUrArCrUrCrUrUrGrUrArGrArUrArArGr | RefSeq26 |
| | | GrArArArUrUrCrGrArArArUrGrArGrUrCrC | |
| 108 | RNP27 | rUrArArUrUrUrCrUrArCrUrCrUrUrGrUrArGrArUrArArAr | RefSeq27 |
| | | CrCrUrGrUrArUrGrArArGrUrUrUrArCrCrU | |
| 109 | RNP28 | rUrArArUrUrUrCrUrArCrUrCrUrUrGrUrArGrArUrArGrAr | RefSeq28 |
| | | GrUrGrGrUrGrArArArCrCrArGrArArCrArA | |
| 110 | RNP29 | rUrArArUrUrUrCrUrArCrUrCrUrUrGrUrArGrArUrArGrAr | RefSeq29 |
| | | UrGrArUrCrArCrArGrUrCrGrUrGrUrUrArA | |

TABLE 7
Exemplary guide RNA (DNA/RNA oligonucleotide)

| SEQ ID | | |
|---|---|---|
| NO. | Component | Details |
| 39 | Target sequence | GAGTGGTGAAACCAGAACAAT |
| 68 | Targeting domain | rGrArGrUrGrGrUrGrArArArCrCrArGrArArCr |
| | (corresponding RNA | ArArU |
| | sequence) | |
| 111 | AsCas12a stem loop | rUrArArUrUrUrCrUrArCrUrCrUrUrGrUrArGr |
| | (scaffold sequence) | ArU |
| 97 | crRNA/gRNA | rUrArArUrUrUrCrUrArCrUrCrUrUrGrUrArGr |
| | | ArUrGr ArGrUrGrGrUrGrArArArCrCrArGrArA |
| | | rCrArArU |
| 112 | gRNA with DNA | ATGTGTTTTTGTCAAAAGACCTTTTrUrArA |
| | extension | rUrUrUrCrUrArCrUrCrUrUrGrUrArGrArUrGr |
| | | ArGrUrGrGrUrGrArArArCrCrArGrArArCrArA |
| | | rU |

All bases are in upper case
Lowercase “r” represents RNA, 2′-hydroxy; bases not modified by an “r” are DNA
All linkages between the nucleotides are standard phosphodiester groups.

gRNA Modifications

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, can be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cas12a gRNA, and/or a targeting domain of a gRNA.

As one example, the 5′ end of a gRNA can include a eukaryotic mRNA cap structure or cap analog (e.g., a G(5′)ppp(5′)G cap analog, a m7G(5)ppp(5′)G cap analog, or a 3′-O-Me-m7G(5)ppp(5′)G anti reverse cap analog (ARCA)), as shown below:

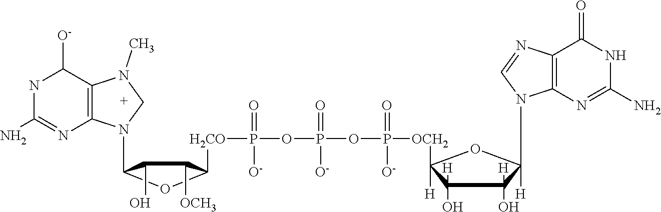

The cap or cap analog can be included during either chemical synthesis or in vitro transcription of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another modification involves the addition, at the 3′ end of a gRNA, of a plurality (e.g., 1-10, 10-20, or 25-200) of adenine (A) residues referred to as a polyA tract. The polyA tract can be added to a gRNA during chemical synthesis, following in vitro transcription using a polyadenosine polymerase (e.g., E. coli Poly(A)Polymerase), or in vivo by means of a polyadenylation sequence, as described in Maeder.

It should be noted that the modifications described herein can be combined in any suitable manner, e.g. a gRNA, whether transcribed in vivo from a DNA vector, or in vitro transcribed gRNA, can include either or both of a 5′ cap structure or cap analog and a 3′ polyA tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

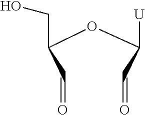

wherein “U” can be an unmodified or modified uridine.

The 3′ terminal U ribose can be modified with a 2′3′ cyclic phosphate as shown below:

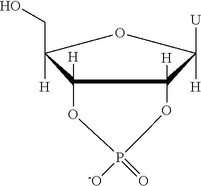

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA are deoxynucleotides.

### RNA-Guided Nucleases

RNA-guided nucleases according to the present disclosure include, but are not limited to, naturally-occurring Class 2 CRISPR nucleases such as Cas9, and Cas12a, as well as other nucleases derived or obtained therefrom. In functional terms, RNA-guided nucleases are defined as those nucleases that: (a) interact with (e.g. complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations can exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cas12a), species (e.g. S. pyogenes vs. S. aureus) or variation (e.g. full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. For example, Cas9 nucleases recognize PAM sequences that are 3′ of the protospacer, while Cas12a, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cas12a recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule can be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389, Sep. 12, 2013 (Ran), incorporated by reference herein), or that that do not cut at all.

### Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g. a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, whereas the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e. bottom) strand of the target nucleic acid. It can be formed from two or more split RuvC motifs (such as RuvC I, RuvCII, and RuvCIII in s. pyogenes and s. aureus). The HNH domain, meanwhile, is structurally similar to HNN endonuclease motifs, and cleaves the complementary (i.e. top) strand of the target nucleic acid. The PI domain, as its name suggests, contributes to PAM specificity.

While certain functions of Cas9 are linked to (but not necessarily fully determined by) the specific domains set forth above, these and other functions can be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

### Cas12a (Formerly Known as Cpf1)

The crystal structure of Acidaminococcus sp. Cas12a in complex with crRNA and a double-stranded (ds) DNA target including a TTTN PAM sequence has been solved by Yamano et al. (Cell. 2016 May 5; 165(4): 949-962 (Yamano), incorporated by reference herein). Cas12a, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cas12a REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cas12a share similarities in structure and function, it should be appreciated that certain Cas12a activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cas12a gRNA (the handle) adopts a pseudoknot structure, rather than a stem loop structure formed by the repeat:antirepeat duplex in Cas9 gRNAs.

### Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that can be made in the RuvC domains, in the Cas9 HNH domain, or in the Cas12a Nuc domain are described in Ran and Yamano, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain or of a Cas9 HNH domain results in a nickase.

Modifications of PAM specificity relative to naturally occurring Cas9 reference molecules has been described by Kleinstiver et al. for both S. pyogenes (Kleinstiver et al., Nature. 2015 Jul. 23; 523(7561):481-5 (Kleinstiver I) and S. aureus (Kleinstiver et al., Nat Biotechnol. 2015 December; 33(12): 1293-1298 (Klienstiver II)). Kleinstiver et al. have also described modifications that improve the targeting fidelity of Cas9 (Nature, 2016 Jan. 28; 529, 490-495 (Kleinstiver III)). Each of these references is incorporated by reference herein.

RNA-guided nucleases have been split into two or more parts, as described by Zetsche et al. (Nat Biotechnol. 2015 February; 33(2):139-42 (Zetsche II), incorporated by reference), and by Fine et al. (Sci Rep. 2015 Jul. 1; 5:10777 (Fine), incorporated by reference).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotechnology 32, 577-582 (2014), which is incorporated by reference for all purposes herein.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications can be possible or desirable in certain applications. For brevity, therefore, exemplary systems, methods and compositions of the present disclosure are presented with reference to particular RNA-guided nucleases, but it should be understood that the RNA-guided nucleases used can be modified in ways that do not alter their operating principles. Such modifications are within the scope of the present disclosure.

Exemplary suitable nuclease variants include, but are not limited to, AsCas12a variants comprising an M537R substitution, an H800A substitution, and/or an F870L substitution, or any combination thereof (numbering scheme according to AsCas12a wild-type sequence). Other suitable modifications of the AsCas12a amino acid sequence are known to those of ordinary skill in the art. Some non-limiting exemplary sequences of wild-type AsCas12a and AsCas12a variants are as follows:

His-AsCas12a-sNLS-sNLS H800A amino acid sequence
[SEQ ID NO: 113]
MGHHHHHHGSTQFEGFTNLYQVSKTLRFELIPQGKTLKHIQEQGFIEEDKARNDH
YKELKPIIDRIYKTYADQCLQLVQLDWENLSAAIDSYRKEKTEETRNALIEEQATY
RNAIHDYFIGRTDNLTDAINKRHAEIYKGLFKAELFNGKVLKQLGTVTTTEHENA
LLRSFDKFTTYFSGFYENRKNVFSAEDISTAIPHRIVQDNFPKFKENCHIFTRLITAV
PSLREHFENVKKAIGIFVSTSIEEVFSFPFYNQLLTQTQIDLYNQLLGGISREAGTEKI
KGLNEVLNLAIQKNDETAHIIASLPHRFIPLFKQILSDRNTLSFILEEFKSDEEVIQSF
CKYKTLLRNENVLETAEALFNELNSIDLTHIFISHKKLETISSALCDHWDTLRNALY
ERRISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIK
LEMEPSLSFYNKARNYATKKPYSVEKFKLNFQMPTLASGWDVNKEKNNGAILFV
KNGLYYLGIMPKQKGRYKALSFEPTEKTSEGFDKMYYDYFPDAAKMIPKCSTQL
KAVTAHFQTHTTPILLSNNFIEPLEITKEIYDLNNPEKEPKKFQTAYAKKTGDQKG
YREALCKWIDFTRDFLSKYTKTTSIDLSSLRPSSQYKDLGEYYAELNPLLYHISFQR
IAEKEIMDAVETGKLYLFQIYNKDFAKGHHGKPNLHTLYWTGLFSPENLAKTSIK
LNGQAELFYRPKSRMKRMAARLGEKMLNKKLKDQKTPIPDTLYQELYDYVNHR
LSHDLSDEARALLPNVITKEVSHEIIKDRRFTSDKFFFHVPITLNYQAANSPSKFNQ
RVNAYLKEHPETPIIGIDRGERNLIYITVIDSTGKILEQRSLNTIQQFDYQKKLDNRE
KERVAARQAWSVVGTIKDLKQGYLSQVIHEIVDLMIHYQAVVVLENLNFGFKSK
RTGIAEKAVYQQFEKMLIDKLNCLVLKDYPAEKVGGVLNPYQLTDQFTSFAKMG
TQSGFLFYVPAPYTSKIDPLTGFVDPFVWKTIKNHESRKHFLEGFDFLHYDVKTGD
FILHFKMNRNLSFQRGLPGFMPAWDIVFEKNETQFDAKGTPFIAGKRIVPVIENHR
FTGRYRDLYPANELIALLEEKGIVFRDGSNILPKLLENDDSHAIDTMVALIRSVLQ
MRNSNAATGEDYINSPVRDLNGVCFDSRFQNPEWPMDADANGAYHIALKGQLLL
NHLKESKDLKLQNGISNQDWLAYIQELRNGSPK KKRKVGSPKKKRKV
Cas12a variant 1 amino acid
[SEQ ID NO: 114]
MTQFEGFTNLYQVSKTLRFELIPQGKTLKHIQEQGFIEEDKARNDHYKELKPIIDRI
YKTYADQCLQLVQLDWENLSAAIDSYRKEKTEETRNALIEEQATYRNAIHDYFIG
RTDNLTDAINKRHAEIYKGLFKAELFNGKVLKQLGTVTTTEHENALLRSFDKFTT
YFSGFYENRKNVFSAEDISTAIPHRIVQDNFPKFKENCHIFTRLITAVPSLREHFENV
KKAIGIFVSTSIEEVFSFPFYNQLLTQTQIDLYNQLLGGISREAGTEKIKGLNEVLNL
AIQKNDETAHIIASLPHRFIPLFKQILSDRNTLSFILEEFKSDEEVIQSFCKYKTLLRN
ENVLETAEALFNELNSIDLTHIFISHKKLETISSALCDHWDTLRNALYERRISELTG
KITKSAKEKVQRSLKHEDINLQEIISAAGKELSEAFKQKTSEILSHAHAALDQPLPT
TLKKQEEKEILKSQLDSLLGLYHLLDWFAVDESNEVDPEFSARLTGIKLEMEPSLS
FYNKARNYATKKPYSVEKFKLNFQRPTLASGWDVNKEKNNGAILFVKNGLYYLG
IMPKQKGRYKALSFEPTEKTSEGFDKMYYDYFPDAAKMIPKCSTQLKAVTAHFQT
HTTPILLSNNFIEPLEITKEIYDLNNPEKEPKKFQTAYAKKTGDQKGYREALCKWID
FTRDFLSKYTKTTSIDLSSLRPSSQYKDLGEYYAELNPLLYHISFQRIAEKEIMDAV
ETGKLYLFQIYNKDFAKGHHGKPNLHTLYWTGLFSPENLAKTSIKLNGQAELFYR
PKSRMKRMAHRLGEKMLNKKLKDQKTPIPDTLYQELYDYVNHRLSHDLSDEAR
ALLPNVITKEVSHEIIKDRRFTSDKFLFHVPITLNYQAANSPSKFNQRVNAYLKEHP
ETPIIGIDRGERNLIYITVIDSTGKILEQRSLNTIQQFDYQKKLDNREKERVAARQA
WSVVGTIKDLKQGYLSQVIHEIVDLMIHYQAVVVLENLNFGFKSKRTGIAEKAVY
QQFEKMLIDKLNCLVLKDYPAEKVGGVLNPYQLTDQFTSFAKMGTQSGFLFYVP
APYTSKIDPLTGFVDPFVWKTIKNHESRKHFLEGFDFLHYDVKTGDFILHFKMNR
NLSFQRGLPGFMPAWDIVFEKNETQFDAKGTPFIAGKRIVPVIENHRFTGRYRDLY
PANELIALLEEKGIVFRDGSNILPKLLENDDSHAIDTMVALIRSVLQMRNSNAATG
EDYINSPVRDLNGVCFDSRFQNPEWPMDADANGAYHIALKGQLLLNHLKESKDL
KLQNGISNQDWLAYIQELRNGRSSDDEATADSQHAAPPKKKRKVGGSGGSGGSG
GSGGSGGSGGSGGSLEHHHHHH
Cas12a variant 2 amino acid sequence
[SEQ ID NO: 115]
MTQFEGFTNLYQVSKTLRFELIPQGKTLKHIQEQGFIEEDKARNDHYKELKPIIDRI
YKTYADQCLQLVQLDWENLSAAIDSYRKEKTEETRNALIEEQATYRNAIHDYFIG
RTDNLTDAINKRHAEIYKGLFKAELFNGKVLKQLGTVTTTEHENALLRSFDKFTT
YFSGFYENRKNVFSAEDISTAIPHRIVQDNFPKFKENCHIFTRLITAVPSLREHFENV
KKAIGIFVSTSIEEVFSFPFYNQLLTQTQIDLYNQLLGGISREAGTEKIKGLNEVLNL
AIQKNDETAHIIASLPHRFIPLFKQILSDRNTLSFILEEFKSDEEVIQSFCKYKTLLRN
ENVLETAEALFNELNSIDLTHIFISHKKLETISSALCDHWDTLRNALYERRISELTG
KITKSAKEKVQRSLKHEDINLQEIISAAGKELSEAFKQKTSEILSHAHAALDQPLPT
TLKKQEEKEILKSQLDSLLGLYHLLDWFAVDESNEVDPEFSARLTGIKLEMEPSLS
FYNKARNYATKKPYSVEKFKLNFQMPTLASGWDVNKEKNNGAILFVKNGLYYL
GIMPKQKGRYKALSFEPTEKTSEGFDKMYYDYFPDAAKMIPKCSTQLKAVTAHF
QTHTTPILLSNNFIEPLEITKEIYDLNNPEKEPKKFQTAYAKKTGDQKGYREALCK
WIDFTRDFLSKYTKTTSIDLSSLRPSSQYKDLGEYYAELNPLLYHISFQRIAEKEIM
DAVETGKLYLFQIYNKDFAKGHHGKPNLHTLYWTGLFSPENLAKTSIKLNGQAEL
FYRPKSRMKRMAHRLGEKMLNKKLKDQKTPIPDTLYQELYDYVNHRLSHDLSDE
ARALLPNVITKEVSHEIIKDRRFTSDKFFFHVPITLNYQAANSPSKFNQRVNAYLKE
HPETPIIGIDRGERNLIYITVIDSTGKILEQRSLNTIQQFDYQKKLDNREKERVAARQ
AWSVVGTIKDLKQGYLSQVIHEIVDLMIHYQAVVVLENLNFGFKSKRTGIAEKAV
YQQFEKMLIDKLNCLVLKDYPAEKVGGVLNPYQLTDQFTSFAKMGTQSGFLFYV
PAPYTSKIDPLTGFVDPFVWKTIKNHESRKHFLEGFDFLHYDVKTGDFILHFKMNR
NLSFQRGLPGFMPAWDIVFEKNETQFDAKGTPFIAGKRIVPVIENHRFTGRYRDLY
PANELIALLEEKGIVFRDGSNILPKLLENDDSHAIDTMVALIRSVLQMRNSNAATG
EDYINSPVRDLNGVCFDSRFQNPEWPMDADANGAYHIALKGQLLLNHLKESKDL
KLQNGISNQDWLAYIQELRNGRSSDDEATADSQHAAPPKKKRKVGGSGGSGGSG
GSGGSGGSGGSGGSLEHHHHHH
Cas12a variant 3 amino acid sequence
[SEQ ID NO: 116]
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFI
GRTDNLTDAINKRHAEIYKGLFKAELFNGKVLKQLGTVTTTEHENALLRSFDKFT
TYFSGFYENRKNVFSAEDISTAIPHRIVQDNFPKFKENCHIFTRLITAVPSLREHFE
NVKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKYKT
LLRNENVLETAEALFNELNSIDLTHIFISHKKLETISSALCDHWDTLRNALYERRIS
ELTGKITKSAKEKVQRSLKHEDINLQEIISAAGKELSEAFKQKTSEILSHAHAALD
QPLPTTLKKQEEKEILKSQLDSLLGLYHLLDWFAVDESNEVDPEFSARLTGIKLE
MEPSLSFYNKARNYATKKPYSVEKFKLNFQRPTLASGWDVNKEKNNGAILFVK
NGLYYLGIMPKQKGRYKALSFEPTEKTSEGFDKMYYDYFPDAAKMIPKCSTQLK
AVTAHFQTHTTPILLSNNFIEPLEITKEIYDLNNPEKEPKKFQTAYAKKTGDQKGY
REALCKWIDFTRDFLSKYTKTTSIDLSSLRPSSQYKDLGEYYAELNPLLYHISFQRI
AEKEIMDAVETGKLYLFQIYNKDFAKGHHGKPNLHTLYWTGLFSPENLAKTSIK
LNGQAELFYRPKSRMKRMAARLGEKMLNKKLKDQKTPIPDTLYQELYDYVNHR
LSHDLSDEARALLPNVITKEVSHEIIKDRRFTSDKFLFHVPITLNYQAANSPSKFN
QRVNAYLKEHPETPIIGIDRGERNLIYITVIDSTGKILEQRSLNTIQQFDYQKKLDN
REKERVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFTSFAK
MGTQSGFLFYVPAPYTSKIDPLTGFVDPFVWKTIKNHESRKHFLEGFDFLHYDVK
TGDFILHFKMNRNLSFQRGLPGFMPAWDIVFEKNETQFDAKGTPFIAGKRIVPVIE
NHRFTGRYRDLYPANELIALLEEKGIVFRDGSNILPKLLENDDSHAIDTMVALIRS
VLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKK
RKVGGSGGSGGSGGSGGSGGSGGSGGSLEHHHHHH
Cas12a variant 4 amino acid sequence
[SEQ ID NO: 117]
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFI
GRTDNLTDAINKRHAEIYKGLFKAELFNGKVLKQLGTVTTTEHENALLRSFDKFT
TYFSGFYENRKNVFSAEDISTAIPHRIVQDNFPKFKENCHIFTRLITAVPSLREHFE
NVKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKYKT
LLRNENVLETAEALFNELNSIDLTHIFISHKKLETISSALCDHWDTLRNALYERRIS
ELTGKITKSAKEKVQRSLKHEDINLQEIISAAGKELSEAFKQKTSEILSHAHAALD
QPLPTTLKKQEEKEILKSQLDSLLGLYHLLDWFAVDESNEVDPEFSARLTGIKLE
MEPSLSFYNKARNYATKKPYSVEKFKLNFQRPTLASGWDVNKEKNNGAILFVK
NGLYYLGIMPKQKGRYKALSFEPTEKTSEGFDKMYYDYFPDAAKMIPKCSTQLK
AVTAHFQTHTTPILLSNNFIEPLEITKEIYDLNNPEKEPKKFQTAYAKKTGDQKGY
REALCKWIDFTRDFLSKYTKTTSIDLSSLRPSSQYKDLGEYYAELNPLLYHISFQRI
AEKEIMDAVETGKLYLFQIYNKDFAKGHHGKPNLHTLYWTGLFSPENLAKTSIK
LNGQAELFYRPKSRMKRMAARLGEKMLNKKLKDQKTPIPDTLYQELYDYVNHR
LSHDLSDEARALLPNVITKEVSHEIIKDRRFTSDKFLFHVPITLNYQAANSPSKFN
QRVNAYLKEHPETPIIGIDRGERNLIYITVIDSTGKILEQRSLNTIQQFDYQKKLDN
REKERVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFTSFAK
MGTQSGFLFYVPAPYTSKIDPLTGFVDPFVWKTIKNHESRKHFLEGFDFLHYDVK
TGDFILHFKMNRNLSFQRGLPGFMPAWDIVFEKNETQFDAKGTPFIAGKRIVPVIE
NHRFTGRYRDLYPANELIALLEEKGIVFRDGSNILPKLLENDDSHAIDTMVALIRS
VLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKK
RKV
Cas12a variant 4 amino acid sequence
[SEQ ID NO: 130]
MTQFEGFTNLYQVSKTLRFELIPQGKTLKHIQEQGFIEEDKARNDHYKELKPIIDRI
YKTYADQCLQLVQLDWENLSAAIDSYRKEKTEETRNALIEEQATYRNAIHDYFIG
RTDNLTDAINKRHAEIYKGLFKAELFNGKVLKQLGTVTTTEHENALLRSFDKFTT
YFSGFYENRKNVFSAEDISTAIPHRIVQDNFPKFKENCHIFTRLITAVPSLREHFENV
KKAIGIFVSTSIEEVFSFPFYNQLLTQTQIDLYNQLLGGISREAGTEKIKGLNEVLNL
AIQKNDETAHIIASLPHRFIPLFKQILSDRNTLSFILEEFKSDEEVIQSFCKYKTLLRN
ENVLETAEALFNELNSIDLTHIFISHKKLETISSALCDHWDTLRNALYERRISELTG
KITKSAKEKVQRSLKHEDINLQEIISAAGKELSEAFKQKTSEILSHAHAALDQPLPT
TLKKQEEKEILKSQLDSLLGLYHLLDWFAVDESNEVDPEFSARLTGIKLEMEPSLS
FYNKARNYATKKPYSVEKFKLNFQRPTLASGWDVNKEKNNGAILFVKNGLYYLG
IMPKQKGRYKALSFEPTEKTSEGFDKMYYDYFPDAAKMIPKCSTQLKAVTAHFQT
HTTPILLSNNFIEPLEITKEIYDLNNPEKEPKKFQTAYAKKTGDQKGYREALCKWID
FTRDFLSKYTKTTSIDLSSLRPSSQYKDLGEYYAELNPLLYHISFQRIAEKEIMDAV
ETGKLYLFQIYNKDFAKGHHGKPNLHTLYWTGLFSPENLAKTSIKLNGQAELFYR
PKSRMKRMAARLGEKMLNKKLKDQKTPIPDTLYQELYDYVNHRLSHDLSDEAR
ALLPNVITKEVSHEIIKDRRFTSDKFLFHVPITLNYQAANSPSKFNQRVNAYLKEHP
ETPIIGIDRGERNLIYITVIDSTGKILEQRSLNTIQQFDYQKKLDNREKERVAARQA
WSVVGTIKDLKQGYLSQVIHEIVDLMIHYQAVVVLENLNFGFKSKRTGIAEKAVY
QQFEKMLIDKLNCLVLKDYPAEKVGGVLNPYQLTDQFTSFAKMGTQSGFLFYVP
APYTSKIDPLTGFVDPFVWKTIKNHESRKHFLEGFDFLHYDVKTGDFILHFKMNR
NLSFQRGLPGFMPAWDIVFEKNETQFDAKGTPFIAGKRIVPVIENHRFTGRYRDLY
PANELIALLEEKGIVFRDGSNILPKLLENDDSHAIDTMVALIRSVLQMRNSNAATG
EDYINSPVRDLNGVCFDSRFQNPEWPMDADANGAYHIALKGQLLLNHLKESKDL
KLQNGISNQDWLAYIQELRN
Cas12a variant 5 amino acid sequence
[SEQ ID NO: 118]
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFI
GRTDNLTDAINKRHAEIYKGLFKAELFNGKVLKQLGTVTTTEHENALLRSFDKFT
TYFSGFYENRKNVFSAEDISTAIPHRIVQDNFPKFKENCHIFTRLITAVPSLREHFE
NVKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKYKT
LLRNENVLETAEALFNELNSIDLTHIFISHKKLETISSALCDHWDTLRNALYERRIS
ELTGKITKSAKEKVQRSLKHEDINLQEIISAAGKELSEAFKQKTSEILSHAHAALD
QPLPTTLKKQEEKEILKSQLDSLLGLYHLLDWFAVDESNEVDPEFSARLTGIKLE
MEPSLSFYNKARNYATKKPYSVEKFKLNFQRPTLASGWDVNKEKNNGAILFVK
NGLYYLGIMPKQKGRYKALSFEPTEKTSEGFDKMYYDYFPDAAKMIPKCSTQLK
AVTAHFQTHTTPILLSNNFIEPLEITKEIYDLNNPEKEPKKFQTAYAKKTGDQKGY
REALCKWIDFTRDFLSKYTKTTSIDLSSLRPSSQYKDLGEYYAELNPLLYHISFQRI
AEKEIMDAVETGKLYLFQIYNKDFAKGHHGKPNLHTLYWTGLFSPENLAKTSIK
LNGQAELFYRPKSRMKRMAHRLGEKMLNKKLKDQKTPIPDTLYQELYDYVNHR
LSHDLSDEARALLPNVITKEVSHEIIKDRRFTSDKFLFHVPITLNYQAANSPSKFN
QRVNAYLKEHPETPIIGIDRGERNLIYITVIDSTGKILEQRSLNTIQQFDYQKKLDN
REKERVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFTSFAK
MGTQSGFLFYVPAPYTSKIDPLTGFVDPFVWKTIKNHESRKHFLEGFDFLHYDVK
TGDFILHFKMNRNLSFQRGLPGFMPAWDIVFEKNETQFDAKGTPFIAGKRIVPVIE
NHRFTGRYRDLYPANELIALLEEKGIVFRDGSNILPKLLENDDSHAIDTMVALIRS
VLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKK
RKV
Cas12a variant 6 amino acid sequence
[SEQ ID NO: 119]
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFI
GRTDNLTDAINKRHAEIYKGLFKAELFNGKVLKQLGTVTTTEHENALLRSFDKFT
TYFSGFYENRKNVFSAEDISTAIPHRIVQDNFPKFKENCHIFTRLITAVPSLREHFE
NVKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKYKT
LLRNENVLETAEALFNELNSIDLTHIFISHKKLETISSALCDHWDTLRNALYERRIS
ELTGKITKSAKEKVQRSLKHEDINLQEIISAAGKELSEAFKQKTSEILSHAHAALD
QPLPTTLKKQEEKEILKSQLDSLLGLYHLLDWFAVDESNEVDPEFSARLTGIKLE
MEPSLSFYNKARNYATKKPYSVEKFKLNFQRPTLASGWDVNKEKNNGAILFVK
NGLYYLGIMPKQKGRYKALSFEPTEKTSEGFDKMYYDYFPDAAKMIPKCSTQLK
AVTAHFQTHTTPILLSNNFIEPLEITKEIYDLNNPEKEPKKFQTAYAKKTGDQKGY
REALCKWIDFTRDFLSKYTKTTSIDLSSLRPSSQYKDLGEYYAELNPLLYHISFQRI
AEKEIMDAVETGKLYLFQIYNKDFAKGHHGKPNLHTLYWTGLFSPENLAKTSIK
LNGQAELFYRPKSRMKRMAHRLGEKMLNKKLKDQKTPIPDTLYQELYDYVNHR
LSHDLSDEARALLPNVITKEVSHEIIKDRRFTSDKFLFHVPITLNYQAANSPSKFN
QRVNAYLKEHPETPIIGIDRGERNLIYITVIDSTGKILEQRSLNTIQQFDYQKKLDN
REKERVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFTSFAK
MGTQSGFLFYVPAPYTSKIDPLTGFVDPFVWKTIKNHESRKHFLEGFDFLHYDVK
TGDFILHFKMNRNLSFQRGLPGFMPAWDIVFEKNETQFDAKGTPFIAGKRIVPVIE
NHRFTGRYRDLYPANELIALLEEKGIVFRDGSNILPKLLENDDSHAIDTMVALIRS
VLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKK
RKVGGSGGSGGSGGSGGSGGSGGSGGSLEHHHHHH
Cas12a variant 7 amino acid sequence
[SEQ ID NO: 120]
MGRDPGKPIPNPLLGLDSTAPKKKRKVGIHGVPAATQFEGFTNLYQVSKTLRFEL
IPQGKTLKHIQEQGFIEEDKARNDHYKELKPIIDRIYKTYADQCLQLVQLDWENL
SAAIDSYRKEKTEETRNALIEEQATYRNAIHDYFIGRTDNLTDAINKRHAEIYKGL
FKAELFNGKVLKQLGTVTTTEHENALLRSFDKFTTYFSGFYENRKNVFSAEDIST
AIPHRIVQDNFPKFKENCHIFTRLITAVPSLREHFENVKKAIGIFVSTSIEEVFSFPFY
NQLLTQTQIDLYNQLLGGISREAGTEKIKGLNEVLNLAIQKNDETAHIIASLPHRFI
PLFKQILSDRNTLSFILEEFKSDEEVIQSFCKYKTLLRNENVLETAEALFNELNSID
LTHIFISHKKLETISSALCDHWDTLRNALYERRISELTGKITKSAKEKVQRSLKHE
DINLQEIISAAGKELSEAFKQKTSEILSHAHAALDQPLPTTLKKQEEKEILKSQLDS
LLGLYHLLDWFAVDESNEVDPEFSARLTGIKLEMEPSLSFYNKARNYATKKPYS
VEKFKLNFQMPTLASGWDVNKEKNNGAILFVKNGLYYLGIMPKQKGRYKALSF
EPTEKTSEGFDKMYYDYFPDAAKMIPKCSTQLKAVTAHFQTHTTPILLSNNFIEPL
EITKEIYDLNNPEKEPKKFQTAYAKKTGDQKGYREALCKWIDFTRDFLSKYTKTT
SIDLSSLRPSSQYKDLGEYYAELNPLLYHISFQRIAEKEIMDAVETGKLYLFQIYN
KDFAKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVITKEV
SHEIIKDRRFTSDKFFFHVPITLNYQAANSPSKFNQRVNAYLKEHPETPIIGIDRGE
RNLIYITVIDSTGKILEQRSLNTIQQFDYQKKLDNREKERVAARQAWSVVGTIKD
LKQGYLSQVIHEIVDLMIHYQAVVVLENLNFGFKSKRTGIAEKAVYQQFEKMLI
DKLNCLVLKDYPAEKVGGVLNPYQLTDQFTSFAKMGTQSGFLFYVPAPYTSKID
PLTGFVDPFVWKTIKNHESRKHFLEGFDFLHYDVKTGDFILHFKMNRNLSFQRGL
PGFMPAWDIVFEKNETQFDAKGTPFIAGKRIVPVIENHRFTGRYRDLYPANELIAL
LEEKGIVFRDGSNILPKLLENDDSHAIDTMVALIRSVLQMRNSNAATGEDYINSP
VRDLNGVCFDSRFQNPEWPMDADANGAYHIALKGQLLLNHLKESKDLKLQNGI
SNQDWLAYIQELRNPKKKRKVKLAAALEHHHHHH
Exemplary AsCas12a wild-type amino acid sequence
[SEQ ID NO: 121]
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFI
GRTDNLTDAINKRHAEIYKGLFKAELFNGKVLKQLGTVTTTEHENALLRSFDKFT
TYFSGFYENRKNVFSAEDISTAIPHRIVQDNFPKFKENCHIFTRLITAVPSLREHFE
NVKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKYKT
LLRNENVLETAEALFNELNSIDLTHIFISHKKLETISSALCDHWDTLRNALYERRIS
ELTGKITKSAKEKVQRSLKHEDINLQEIISAAGKELSEAFKQKTSEILSHAHAALD
QPLPTTLKKQEEKEILKSQLDSLLGLYHLLDWFAVDESNEVDPEFSARLTGIKLE
MEPSLSFYNKARNYATKKPYSVEKFKLNFQMPTLASGWDVNKEKNNGAILFVK
NGLYYLGIMPKQKGRYKALSFEPTEKTSEGFDKMYYDYFPDAAKMIPKCSTQLK
AVTAHFQTHTTPILLSNNFIEPLEITKEIYDLNNPEKEPKKFQTAYAKKTGDQKGY
REALCKWIDFTRDFLSKYTKTTSIDLSSLRPSSQYKDLGEYYAELNPLLYHISFQRI
AEKEIMDAVETGKLYLFQIYNKDFAKGHHGKPNLHTLYWTGLFSPENLAKTSIK
LNGQAELFYRPKSRMKRMAHRLGEKMLNKKLKDQKTPIPDTLYQELYDYVNHR
LSHDLSDEARALLPNVITKEVSHEIIKDRRFTSDKFFFHVPITLNYQAANSPSKFNQ
RVNAYLKEHPETPIIGIDRGERNLIYITVIDSTGKILEQRSLNTIQQFDYQKKLDNR
EKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAK
MGTQSGFLFYVPAPYTSKIDPLTGFVDPFVWKTIKNHESRKHFLEGFDFLHYDVK
TGDFILHFKMNRNLSFQRGLPGFMPAWDIVFEKNETQFDAKGTPFIAGKRIVPVIE
NHRFTGRYRDLYPANELIALLEEKGIVFRDGSNILPKLLENDDSHAIDTMVALIRS
VLQMRNSNAATGEDYINSPVRDLNGVCFDSRFQNPEWPMDADANGAYHIALKG
QLLLNHLKESKDLKLQNGISNQDWLAYIQELRN

In some embodiments, an RNA-guided nuclease has at least 80%, at least 85%, at least 86%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity relative to a wild-type RNA-guided nuclease and/or an RNA-guided nuclease disclosed herein (e.g., an RNA-guided nuclease comprising an amino acid sequence selected from the group consisting of SEQ ID NOs: 113-121 and SEQ ID NO: 130). In some embodiments, an RNA-guided nuclease has 1, 2, 3, 4, 5, 6, 7, 8, 9, 10, 11, 12, 13, 14, 15, 16, 17, 18, 19, or 20 mutations relative to a wild-type RNA-guided nuclease and/or an RNA-guided nuclease disclosed herein (e.g., an RNA-guided nuclease comprising an amino acid sequence selected from the group consisting of SEQ ID NOs: 113-121 and SEQ ID NO: 130). In some embodiments, an RNA-guided nuclease has less than 2, 3, 4, 5, 6, 7, 8, 9, 10, 11, 12, 13, 14, 15, 16, 17, 18, 19, or 20 mutations relative to a wild-type RNA-guided nuclease and/or an RNA-guided nuclease disclosed herein (e.g., an RNA-guided nuclease comprising an amino acid sequence selected from the group consisting of SEQ ID NOs: 113-121 and SEQ ID NO: 130).

### Nucleic Acids Encoding RNA-Guided Nucleases

Nucleic acids encoding RNA-guided nucleases, e.g., Cas9, Cas12a or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, the nucleic acid encoding the RNA-guided nuclease is an RNA. In certain embodiments, the nucleic acid encoding the RNA-guided nuclease is an mRNA. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease can comprise a nucleic acid encoding a nuclear localization sequence (NLS). Nuclear localization sequences are known in the art.

### Functional Analysis of Candidate Molecules

Candidate RNA-guided nucleases, gRNAs, and complexes thereof, can be evaluated by standard methods known in the art. See, e.g. Cotta-Ramusino. The stability of RNP complexes can be evaluated by differential scanning fluorimetry, as described below.

### Differential Scanning Fluorimetry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g. different stoichiometric ratios of gRNA:RNA-guided nuclease protein, different buffer solutions, etc.) to identify optimal conditions for RNP formation; and (b) testing modifications (e.g. chemical modifications, alterations of sequence, etc.) of an RNA-guided nuclease and/or a gRNA to identify those modifications that improve RNP formation or stability. One readout of a DSF assay is a shift in melting temperature of the RNP complex; a relatively high shift suggests that the RNP complex is more stable (and can thus have greater activity or more favorable kinetics of formation, kinetics of degradation, or another functional characteristic) relative to a reference RNP complex characterized by a lower shift. When the DSF assay is deployed as a screening tool, a threshold melting temperature shift can be specified, so that the output is one or more RNPs having a melting temperature shift at or above the threshold. For instance, the threshold can be 5-10° C. (e.g. 5°, 6°, 7° 8°, 9° 10°) or more, and the output can be one or more RNPs characterized by a melting temperature shift greater than or equal to the threshold.

Two non-limiting examples of DSF assay conditions are set forth below:

To determine the best solution to form RNP complexes, a fixed concentration (e.g. 2 μM) of RNA-guided nuclease (e.g., Cas9 or Cas12a) in water+10× SYPRO Orange® (Life Technologies cat #S-6650) is dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay involves mixing various concentrations of gRNA with a fixed concentration (e.g. 2 μM) of RNA-guided nuclease (e.g., Cas9 or Cas12a) in optimal buffer from assay 1 above and incubating (e.g. at RT for 10′) in a 384 well plate. An equal volume of optimal buffer+10× SYPRO Orange® (Life Technologies cat #S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### Genome Editing Strategies

The genome editing systems described above are used, in various embodiments of the present disclosure, to generate edits in (i.e. to alter) targeted regions of DNA within or obtained from a cell. Various strategies are described herein to generate particular edits, and these strategies are generally described in terms of the desired repair outcome, the number and positioning of individual edits (e.g. SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs can result in other outcomes. The description of a particular editing strategy or method should not be understood to require a particular repair outcome unless otherwise specified.

Replacement of a targeted region in certain embodiments involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g. a homologous sequence within the cellular genome), to promote gene conversion. Exogenous templates can have asymmetric overhangs (i.e. the portion of the template that is complementary to the site of the DSB can be offset in a 3′ or 5′ direction, rather than being centered within the donor template), for instance as described by Richardson et al. (Nature Biotechnology 34, 339-344 (2016), (Richardson), incorporated by reference). In instances where the template is single stranded, it can correspond to either the complementary (top) or non-complementary (bottom) strand of the targeted region.

Gene conversion and gene correction are facilitated, in some cases, by the formation of one or more nicks in or around the targeted region, as described in Ran and Cotta-Ramusino. In some cases, a dual-nickase strategy is used to form two offset SSBs that, in turn, form a single DSB having an overhang (e.g. a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends can be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g. ±1, ±2, ±3, etc. As one example, in a lead-finding setting, multiple gRNAs can be screened to identify those gRNAs that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

### Multiplex Strategies

While exemplary strategies discussed above have focused on repair outcomes mediated by single DSBs, genome editing systems according to this disclosure can also be employed to generate two or more DSBs, either in the same locus or in different loci. Strategies for editing that involve the formation of multiple DSBs, or SSBs, are described in, for instance, Cotta-Ramusino.

In certain embodiments, the present disclosure provides an isolated T cell or population of T cells that include a modification, e.g., disruption, in two or more endogenous genes of a T cell. In certain embodiments, such modification is introduced into the T cell or population of T cells by one or more genome editing systems described herein. In certain embodiments, the instant disclosure relates to the use of a genome editing system to edit a target PTPN2 target nucleic acid sequence and one or more additional endogenous genes of a T cell. For example, the additional endogenous gene can be selected from the group consisting of FAS, BID, CTLA4, PDCD1, CBLB, PTPN6, B2M, TRAC, CIITA, TRBC and any combination thereof. For example, but not by way of limitation, multiple modifications in the genome of a T cell can be generated by the delivery of two or more complexes comprising an RNA-guided nuclease (e.g. Cas9 and/or Cas12a) and a gRNA molecule, e.g., RNP complexes, that target a PTPN2 gene sequence and one or more of a FAS gene sequence, BID gene sequence, CTLA4 gene sequence, PDCD1 gene sequence, CBLB gene sequence, PTPN6 gene sequence, B2M gene sequence, TRAC gene sequence, CIITA gene sequence, TRBC gene sequence or a combination thereof. For example, and not by way of limitation, two or more, three or more, four or more, five or more, six or more, seven or more, eight or more, nine or more or ten complexes, e.g., RNP complexes, can be delivered, where each of the complexes target a different gene. In certain embodiments, the gRNA can be complementary to either strand of the gene to be targeted. In certain embodiments, the gRNA molecule can target a regulatory region, an intron or an exon of the gene to be targeted. In certain embodiments, the genome editing system comprises a gRNA complementary to a PTPN2 target nucleic acid sequence and a gRNA complementary to a target nucleic acid sequence of one or more additional endogenous genes selected from the group consisting of FAS, BID, CTLA4, PDCD1, CBLB, PTPN6, B2M, TRAC, CIITA, TRBC, and a combination thereof. In certain embodiments, the gRNA can be complementary to either strand of the additional endogenous gene. In certain embodiments, the targeted portion of the additional endogenous gene is within the coding sequence of the additional endogenous gene. In certain embodiments, the targeted portion of the additional endogenous gene is within an exon. In certain embodiments, the targeted portion of the additional endogenous gene is within an intron. In certain embodiments, the targeted portion of the additional endogenous gene is within a regulatory region of the gene. In certain embodiments, more than one sequence of an additional endogenous gene is targeted and the targeted portions of the additional endogenous gene are within one or more exons, one or more introns, one or more regulatory regions or one or more exons, one or more introns and one or more regulatory regions. In certain embodiments, the sequence of one or more gRNAs targeting an additional endogenous gene, or of one or more target DNA sequences of the additional endogenous gene, are set forth in the International Patent Publication No. WO 2019/118516 or the International Publication No. WO 2015/161276, both of which are incorporated by reference herein in their entireties.

### Donor Template Design

Donor template design is described in detail in the literature, for instance in Cotta-Ramusino. DNA oligomer donor templates (oligodeoxynucleotides or ODNs), which can be single stranded (ssODNs) or double-stranded (dsODNs), can be used to facilitate HDR-based repair of DSBs, and are particularly useful for introducing alterations into a target DNA sequence, inserting a new sequence into the target sequence, or replacing the target sequence altogether.

Whether single-stranded or double stranded, donor templates generally include regions that are homologous to regions of DNA within or near (e.g. flanking or adjoining) a target sequence to be cleaved. These homologous regions are referred to here as “homology arms,” and are illustrated schematically below:

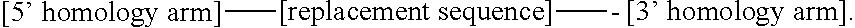

The homology arms can have any suitable length (including 0 nucleotides if only one homology arm is used), and 3′ and 5′ homology arms can have the same length, or can differ in length. The selection of appropriate homology arm lengths can be influenced by a variety of factors, such as the desire to avoid homologies or microhomologies with certain sequences such as Alu repeats or other elements. For example, a 5′ homology arm can be shortened to avoid a sequence repeat element. In other embodiments, a 3′ homology arm can be shortened to avoid a sequence repeat element. In certain embodiments, both the 5′ and the 3′ homology arms can be shortened to avoid including certain sequence repeat elements. In addition, some homology arm designs can improve the efficiency of editing or increase the frequency of a desired repair outcome. For example, Richardson et al. Nature Biotechnology 34, 339-344 (2016) (Richardson), which is incorporated by reference, found that the relative asymmetry of 3′ and 5′ homology arms of single stranded donor templates influenced repair rates and/or outcomes.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One exemplary sequence modification involves the alteration of the naturally-occurring sequence to repair a mutation that is related to a disease or condition of which treatment is desired. Another exemplary sequence modification involves the alteration of one or more sequences that are complementary to, or code for, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN can have any suitable length, e.g., about, at least, or no more than 150-200 nucleotides (e.g., 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can include other coding or non-coding elements. For example, a template nucleic acid can be delivered as part of a viral genome (e.g. in an AAV or lentiviral genome) that includes certain genomic backbone elements (e.g. inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

### Quantitative Measurements of On-Target and Off-Tar Get Gene Editing

It should be noted that the genome editing systems of the present disclosure allow for the detection and quantitative measurement of on-target and off-target gene editing outcomes. The compositions and methods described herein can rely on the use of PCR primer sequences to amplify the genomic locus comprising the expected cut site of the RNA-guided nuclease. In some embodiments, the primers include an adaptor tail for use in a two-step PCR amplification process to prepare amplicon libraries for Next Generation Sequencing (NGS) analysis. A non-limiting example for primers and amplification site for assessing the on-target genome editing efficiency of a PTPN2 target site set forth in [SEQ ID NO: 39] are found in Table 8.

TABLE 8
Exemplary Primer and Amplicon Sequences for an On-Target cut site
analysis

| SEQ ID | | |
|---|---|---|
| NO. | Description | Sequence |
| 122 | Forward | acactctttccctacacgacgctcttccgatctCTCACTTTAAACA |
| | Primer | AGAAATTGAGAAATGAAGC |
| 123 | Reverse | gtgactggagttcagacgtgtgctcttccgatctGGTAACAGATTA |
| | Primer | GTAGCTGAGAACC |
| 124 | Amplicon | ACACTCTTTCCCTACACGACGCTCTTCCGATCTCTCACTTT |
| | | AAACAAGAAATTGAGAAATGAAGCTGGTGATTCAGGGAC |
| | | TCCAAAATCTGGCCAGGTAGTATAATGAAAGTGAGATATT |
| | | GTTCTGGTTTCACCACTCTAAAAAGTGAAAATCAAGGGAG |
| | | AAGTTAGTTCTAACTTTTTTCTTTTAAAAAACTACTTCAGA |
| | | AAGATCATGTGTTTTATATAATTCTTTAAAGCCATATAGGC |
| | | TGTTTATACTATGATTTTTCCATTTGTTTTCTGACAATCCCA |
| | | TATACAAACTAGGGAAGATTTTCTTTAGTGGAAATTTGTG |
| | | ATGTATTAATAATAGCTAACGCATACAACACTTACTATTTT |
| | | GGAGGCCTGTTCTAAGCACTTTACTTATTAACTCAATCCAC |
| | | ACAATAATCCTATTAAAGCCCATTTTCCTGATGAGGAAAC |
| | | TGAAACAAAGGGTAAGATAGTTTGCCCAAGGTCACACAG |
| | | CTGGAACATGGCAGAACCAGGATTTGAAGCAACCTGGGG |
| | | CAGCCTTTGGGTTCTCAGCTACTAATCTGTTACCAGATCGG |
| | | AAGAGCACACGTCTGAACTCCAGTCAC |

Lowercase letters represent partial Illumina adapter tails which are used as round 2 priming sites to amplify the full-length Illumina adapter sequence.

In certain embodiments, the RNPs disclosed herein have minimal or no off-target effects. In certain embodiments, the off-target effect of an RNP is measured by Digenome-seq analysis (Kim et al., Nature Methods (2015); 12:237-243). In certain embodiments, the off-target effect of an RNA is indicated by an off-target count as measured by Digenome-seq analysis. In certain embodiments, the off-target count is measured by Digenome-seq analysis at 1000 nM of the RNP. In certain embodiments, the off-target count is measured by the Digenome-seq analysis at 100 nM of the RNP.

In certain embodiments, the off-target count as measured Digenome-seq analysis of the RNPs disclosed herein at 1000 nM is less than about 20, less than about 19, less than about 18, less than about 17, less than about 16, less than about 15, less than about 14, less than about 13, less than about 12, less than about 11, less than about 10, less than about 9, less than about 8, less than about 7, less than about 6, less than about 5, less than about 4, less than about 3, less than about 2, or less than about 1. In certain embodiments, the off-target count of the RNPs disclosed herein as measured Digenome-seq at 1000 nM is zero or is about zero. In certain embodiments, the off-target count of the RNPs disclosed herein as measured Digenome-seq at 100 nM is less than about 20, less than about 19, less than about 18, less than about 17, less than about 16, less than about 15, less than about 14, less than about 13, less than about 12, less than about 11, less than about 10, less than about 9, less than about 8, less than about 7, less than about 6, less than about 5, less than about 4, less than about 3, less than about 2, or less than about 1. In certain embodiments, the off-target count of the RNPs disclosed herein as measured Digenome-seq at 100 nM is zero or is about zero.

In some embodiments, a quantitative method of assessing on-target and off-target sites includes the integration of an exogenous double stranded oligo nucleotide (dsODN) tag into the genome. For example, GUIDE-Seq (Tsai et al., 2016; Tsai et al., 2014; Tycko et., 2016, which are incorporated by reference herein in their entirety), describe compositions and methods which allow for the quantitative analysis of off-target and on-target gene editing outcomes, by the integration of a dsODN into RNA guided nuclease (RGN) induced double strand breaks (DSBs). In some embodiments, the dsODN tag is a 34 bp, blunt, 5′ phosphorylated, phosphorothioate linked polynucleotide that becomes incorporated into double stand breaks. The dsODN tag contains a priming site that allows for the amplification, sequencing, and discovery of RNG induced double strand breaks. Non-liming examples of the dsODN tag and primer are set forth in Table 9.

TABLE 9
Exemplary Primer and Amplicon Sequences for an Off-Target cut site
analysis

| SEQ ID | | |
|---|---|---|
| NO. | Description | Sequence |
| 125 | dsODN tag | 5′-P-G*T*TTAATTGAGTTGTCATATGTTAATAACGGT*A*T-3′ |
| | (top) | |
| 126 | dsODN tag | 5′-P-A*T*ACCGTTATTAACATATGACAACTCAATTAA*A*C-3′ |
| | (bottom) | |
| 127 | P5 primer | AATGATACGGCGACCACCGAGATCTACAC |
| 128 | dsODN tag | GTGACTGGAGTTCAGACGTGTGCTCTTCCGATCTATACCGTT |
| | specific | ATTAACATATGACAACTCAATTAAAC |
| | primer | |
| | (minus) | |
| 129 | dsODN tag | GTGACTGGAGTTCAGACGTGTGCTCTTCCGATCTGTTTAATT |
| | specific | GAGTTGTCATATGTTAATAACGGTAT |
| | primer (plus) | |

P represents a 5' phosphorylation and * indicates a phosphorothioate linkage.

### Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. The genome editing systems according to this disclosure can incorporate multiple gRNAs, multiple RNA-guided nucleases, and other components such as proteins, and a variety of implementations will be evident to the skilled artisan based on the principles illustrated in systems of the disclosure. In some embodiments the genome editing system of the disclosure are delivered into cells as a ribonucleoprotein (RNP) complex. In some embodiments, one or more RNP complexes are delivered to the cell sequentially in any order, or simultaneously. Tables 10 and 11 set forth several, non-limiting examples of genome editing system implementations. Those of skill in the art will appreciate, however, that these listings are not comprehensive, and that other implementations are possible. With reference to Table 10 in particular, the table lists several exemplary implementations of a genome editing system comprising a single gRNA and an optional donor template. However, genome editing systems according to this disclosure can incorporate multiple gRNAs, multiple RNA-guided nucleases, and other components such as proteins, and a variety of implementations will be evident to the skilled artisan based on the principles illustrated in the table. In the table, [N/A] indicates that the genome editing system does not include the indicated component.

TABLE 10
Genome Editing System Components

| RNA- | | | |
|---|---|---|---|
| guided | | Donor | |
| Nuclease | gRNA | Template | Comments |
| Protein | RNA | [N/A] | An RNA-guided nuclease protein |
| | | | complexed with a gRNA molecule (an |
| | | | RNP complex) |
| Protein | RNA | DNA | An RNP complex as described above |
| | | | plus a single-stranded or double |
| | | | stranded donor template. |
| Protein | DNA | [N/A] | An RNA-guided nuclease protein plus |
| | | | gRNA transcribed from DNA. |
| Protein | DNA | DNA | An RNA-guided nuclease protein plus |
| | | | gRNA-encoding DNA and a separate |
| | | | DNA donor template. |
| Protein | DNA | | An RNA-guided nuclease protein and |
| | | | a single DNA encoding both a gRNA |
| | | | and a donor template. |
| DNA | | | A DNA or DNA vector encoding an |
| | | | RNA-guided nuclease, a gRNA and a |
| | | | donor template. |
| DNA | DNA | [N/A] | Two separate DNAs, or two separate |
| | | | DNA vectors, encoding the RNA- |
| | | | guided nuclease and the gRNA, |
| | | | respectively. |
| DNA | DNA | DNA | Three separate DNAs, or three |
| | | | separate DNA vectors, encoding the |
| | | | RNA-guided nuclease, the gRNA and |
| | | | the donor template, respectively. |
| DNA | | [N/A] | A DNA or DNA vector encoding an |
| | | | RNA-guided nuclease and a gRNA |
| DNA | | DNA | A first DNA or DNA vector encoding |
| | | | an RNA-guided nuclease and a gRNA, |
| | | | and a second DNA or DNA vector |
| | | | encoding a donor template. |
| DNA | DNA | | A first DNA or DNA vector encoding |
| | | | an RNA-guided nuclease and second |
| | | | DNA or DNA vector encoding a |
| | | | gRNA and a donor template. |
| DNA | | | A first DNA or DNA vector encoding |
| | DNA | | an RNA-guided nuclease and a donor |
| | | | template, and a second DNA or DNA |
| | | | vector encoding a gRNA |
| DNA | | | A DNA or DNA vector encoding an |
| | RNA | | RNA-guided nuclease and a donor |
| | | | template, and a gRNA |

| RNA | [N/A] | An RNA or RNA vector encoding an |
|---|---|---|
| | | RNA-guided nuclease and comprising |
| | | a gRNA |
| RNA | DNA | An RNA or RNA vector encoding an |

RNA-guided nuclease and comprising
a gRNA, and a DNA or DNA vector
encoding a donor template.

Table 10 summarizes various delivery methods for the components of genome editing systems, as described herein. Again, the listing is intended to be exemplary rather than limiting.

TABLE 11

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (e.g., electroporation, | | YES | Transient | NO | Nucleic Acids |
| particle gun, Calcium | | | | | and Proteins |
| Phosphate transfection, cell | | | | | |
| compression or squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Stable | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

### Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector based methods (e.g., using naked DNA or DNA complexes), or a combination thereof. In some embodiments the genome editing system of the disclosure are delivered by AAV.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs, T cells). Nucleic acid vectors, such as the vectors summarized in Table 11, can also be used. In some embodiments the genome editing system of the disclosure are delivered into cells by electroporation.

One approach for cell therapy processes includes the direct delivery of active proteins into human cells. A protein delivery agent, the Feldan Shuttle, is a protein-based delivery agent, which is designed for cell therapy (Del'Guidice et al., PLoSOne. 2018 Apr. 4; 13(4):e0195558; incorporated in its entirety herein by reference). In some embodiments the genome editing system of the disclosure are delivered into cells by the Feldan Shuttle.

Nucleic acid vectors can comprise one or more sequences encoding genome editing system components, such as an RNA-guided nuclease, a gRNA and/or a donor template. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vector can include an RNA-guided nuclease (e.g., Cas9 or Cas12a) coding sequence that includes one or more nuclear localization sequences (e.g., a nuclear localization sequence from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino.

Nucleic acid vectors according to this disclosure include recombinant viral vectors. Exemplary viral vectors are set forth in Table 11, and additional suitable viral vectors and their use and production are described in Cotta-Ramusino. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art, and are summarized in Cotta-Ramusino. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 12, and Table 13 lists exemplary polymers for use in gene transfer and/or nanoparticle formulations.

TABLE 12
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)propyl]N,N,N-trimethylammonium chloride | DOTMA | Cationic |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N-dimethyl-1- | DOSPA | Cationic |
| propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]-dimethylammonium | CLIP-1 | Cationic |
| chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14- | Cationic |
| | amidine | |
| Octadecenolyoxy[ethyl-2-heptadecenyl-3 hydroxyethyl] imidazolinium | DOTIM | Cationic |
| chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

TABLE 13
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In certain embodiments, the nucleic acid molecule is delivered at the same time as one or more of the components of the genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the genome editing system are delivered. In certain embodiments, the nucleic acid molecule is delivered by a different means than one or more of the components of the genome editing system, e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

### Delivery of RNPs and or RNA Encoding Genome Editing System Components

RNPs (complexes of gRNAs and RNA-guided nucleases) and/or RNAs encoding RNA-guided nucleases and/or gRNAs, can be delivered into cells or administered to subjects by art-known methods, some of which are described in Cotta-Ramusino. In vitro, RNA-guided nuclease-encoding and/or gRNA-encoding RNA can be delivered, e.g., by microinjection, electroporation, transient cell compression or squeezing (see, e.g., Lee 2012). Lipid-mediated transfection, peptide-mediated delivery, GalNAc- or other conjugate-mediated delivery, and combinations thereof, can also be used for delivery in vitro and in vivo.

In vitro, delivery via electroporation comprises mixing the cells with the RNA encoding RNA-guided nucleases and/or gRNAs, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure.

In certain embodiments, the ribonucleoprotein (RNP) complexes, comprise guide RNAs, Cas12a proteins, including modified Cas12a proteins (AsCas12a variants). Non-limiting examples of Cas12a proteins are set forth in SEQ ID NOs: 113-121 and SEQ ID NO:130. In certain embodiments, an RNP complex may include a guide RNA (gRNA) complexed to a Cas12a protein or a modified Cas12a protein. In certain embodiments a gRNA may comprise a sequence set forth in SEQ ID NOs: 53-81 or SEQ ID NOs: 82-110. In certain embodiments, the RNP complex may comprise an RNP complex set forth in Table 14. For example, an RNP complex may include a gRNA comprising a sequence set forth in SEQ ID NOs: 68, 97, or 112, a modified Cas12a protein set forth in SEQ ID NO: 117 or SEQ ID NO: 130, and target a PTPN2 gene at the sequence set forth in SEQ ID NO: 39 or SEQ ID NO: 131.

TABLE 14
Exemplary ribonucleoprotein (RNP) Configuration

| Component | Details |
|---|---|
| RNP30 | Complex of AsCas12a protein and crRNA/gRNA that targets the site set |
| | forth in [SEQ ID NO: 39] |
| AsCas12a protein | AsCas12a protein with 3 amino acid changes to increase activity and |
| | inactivate RNase activity; C-terminal linker and nuclear localization |
| | sequence added with sequence set forth in [SEQ ID NO: 117] |
| crRNA/gRNA | DNA/RNA oligonucleotide with the sequence set forth in [SEQ ID NO: |
| | 112] |
| PTPN2 target site | 21-base pair site in exon 6 of PTPN2, corresponding to chr18: 12817345- |
| | 12817366 with the sequence set forth in [SEQ ID NO: 39] |

Coordinates based on hg38 (GRCh38) genome with 0-start half-open (also known as 0-based) system

### Target Cells

Genome editing systems according to this disclosure can be used to manipulate or alter a target cell, e.g., to edit or alter a target nucleic acid. The manipulating can occur, in various embodiments, in vivo or ex vivo.

In certain embodiments, the target cell comprises an edit in the target sequence of the PTPN2 gene. In certain embodiments, the target cell comprises an indel in the target sequence of the PTPN2 gene. In certain embodiments, the target cell comprises a deletion of all or a portion of the target sequence of the PTPN2 gene. In certain embodiments, at least 50%, at least 55%, at least 60%, at least 65%, at least 70%, at least 75%, at least 80%, at least 85%, at least 90%, at least 95%, at least 99%, or at least 100% of the target cells contacted with the genome editing system comprise an indel in the PTPN2 gene. An indel may be detected by any method known in the art, e.g., by Illumina amplicon-based sequencing as described in Example 1 herein.

In certain embodiments, the percentage of target cells comprising an indel increases in a concentration dependent manner with respect to increasing the concentration of the RNP complex.

In certain embodiments, the RNP complex induces an indel within or near the target site of a PTPN2 gene with an EC50 value of less than about 5 nM, less than about 6 nM, less than about 7 nM, less than about 8 nM, less than about 9 nM, less than about 10 nM, less than about 11 nM, less than about 12 nM, less than about 12 nM, less than about 13 nM, less than about 14 nM, less than about 15 nM, less than about 16 nM, less than about 17 nM, less than about 18 nM, less than about 18 nM, less than 19 nM, less than about 20 nM, less than about 21 nM, less than about 22 nM, less than about 23 nM, less than about 24 nM, less than about 25 nM, less than about 26 nM, less than about 27 nM, less than about 28 nM, less than about 29 nM, or less than about 30 nM.

In certain embodiments, the level of a PTPN2 gene product is reduced in the target cell relative to a cell that has not been contacted with genome editing system. In certain embodiments, the PTPN2 gene product is a mRNA. In certain embodiments, the PTPN2 gene product is a protein. In certain embodiments, the PTPN2 protein is reduced by at least about 40%, at least about 50%, at least about 60%, at least about 70%, at about least 80%, at least about 90%, at least about 95%, at least about 99%, or at least about 100% in a target cell that has been contacted with the genome editing system relative to cell that has not been contacted with the genome editing system. An amount of PTPN2 protein may be measured by any method known in the art, e.g., by Western blot as described in Example 1 herein.

In certain embodiments, the relative PTPN2 gene product is reduced in a concentration dependent manner with respect to increasing the concentration of the RNP complex. In certain embodiments, the level of the PTPN2 gene product is reduced with respect to the concentration of the RNP complex with an EC50 value of less than about 5 nM, less than about 6 nM, less than about 7 nM, less than about 8 nM, less than about 9 nM, less than about 10 nM, less than about 11 nM, less than about 12 nM, less than about 12 nM, less than about 13 nM, less than about 14 nM, less than about 15 nM, less than about 16 nM, less than about 17 nM, less than about 18 nM, less than about 18 nM, less than about 19 nM, less than about 20 nM, less than about 21 nM, less than about 22 nM, less than about 23 nM, less than about 24 nM, less than about 25 nM, less than about 26 nM, less than about 27 nM, less than about 28 nM, less than about 29 nM, or less than about 30 nM.

In certain embodiments, the target cell is an immunocompetent cell, e.g., a T cell, a CD8+ T cell (e.g., a CD8+ naive T cell, central memory T cell, or effector memory T cell), a CD4+ T cell, an alpha/beta T cell, a gamma/delta T cell, a natural killer T cell (NKT cells), a regulatory T cell (Treg), a stem cell memory T cell, a lymphoid progenitor cell a hematopoietic stem cell, a natural killer cell (NK cell), or a dendritic cell. In certain embodiments, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In certain embodiments, the target cell has been altered to contain specific T cell receptor (TCR) genes (e.g., a TRAC and TRBC gene). In another embodiment, the TCR has binding specificity for a tumor associated antigen. In certain embodiments, the TCR is an engineered TCR.

In certain embodiments, the target cell has been altered to contain a specific chimeric antigen receptor (CAR). In an embodiment, the CAR has binding specificity for a tumor associated antigen.

In another embodiment, the target cell has been altered to bind a tumor antigen, e.g., by a TCR or a CAR.

In certain embodiments, the target cell comprises a genomic edit that results in loss of function of PTPN2. While not wishing to be bound by any particular theory, it is contemplated that disabling PTPN2 signaling will enhance T cell meditated anti-tumor immunity and CAR T-cell therapy against solid tumors. In some embodiments, the target cell is for use in the treatment of a disease, disorder, or condition, e.g., a tumor and/or a cancer.

Provided herein are methods of administering cells and compositions described herein, and uses of such cells and compositions to treat or prevent diseases, conditions, and disorders, including cancers. In some embodiments, the cells and compositions are administered to a subject or patient having the particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered in a therapeutically effective amount to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing, are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically similar. In some embodiments, the second subject expresses the same HLA class or super-type as the first subject.

Among the diseases, conditions, and disorders for treatment with the provided compositions, cells, methods and uses are tumors, including solid tumors, hematologic malignancies, and melanomas, and infectious diseases, such as infection with a virus or other pathogen, e.g., HIV, HCV, HBV, CMV, and parasitic disease. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), acute—lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin, melanoma, bone, and brain cancer, ovarian cancer, epithelial cancers, renal cell carcinoma, pancreatic adenocarcinoma, Hodgkin's lymphoma, cervical carcinoma, colorectal cancer, glioblastoma, neuroblastoma, Ewing's sarcoma, medulloblastoma, osteosarcoma, synovial sarcoma, and/or mesothelioma.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

### EXEMPLARY EMBODIMENTS

A1. The presently disclosed subject matter provides a genome editing system comprising: (a) a gRNA molecule comprising a targeting domain that targets a target sequence of a Protein Tyrosine Phosphatase Non-Receptor Type 2 (PTPN2) gene, and (b) an RNA-guided nuclease, or a nucleic acid encoding the RNA-guided nuclease.

A2. The genome editing system of A1, wherein the target sequence of a PTPN2 gene is in exon 6 of PTPN2.

A3. The genome editing system of A1, wherein (a) the target sequence of a PTPN2 gene comprises a nucleotide sequence selected from the group consisting of SEQ ID NOs: 24-52; and/or (b) the targeting domain comprises a nucleotide sequence selected from the group consisting of SEQ ID NOs: 53-81.

A4. The genome editing system of A2, wherein (a) the target sequence of a PTPN2 gene comprises a nucleotide sequence selected from the group consisting of SEQ ID NO: 29; SEQ ID NO:36; SEQ ID NO: 39; and SEQ ID NO: 51; and/or (b) the targeting domain comprises a nucleotide sequence selected from the group consisting of SEQ ID NO: 58; SEQ ID NO: 65; SEQ ID NO: 68; and SEQ ID NO: 73.

A5. The genome editing system of any one of A1-A4, wherein (a) the target sequence of a PTPN2 gene comprises the nucleotide sequence set forth in SEQ ID NO: 39; and/or (b) the targeting domain comprises the nucleotide sequence set forth in SEQ ID NO: 68.

A6. The genome editing system of any one of A1-A5, wherein the RNA-guided nuclease is selected from the group consisting of Cas9 (e.g., SpCas9, SaCas9, (KKH) SaCas9, eSpCas9, Cas9-HF1, HypaCas9, dCas9-Fokl, Sniper-Cas9, xCas9, evoCas9, SpCas9-NG, VRQR, VRER, NmeCas9, CjCas9), Cas12, Cas12a (also known as Cpf1; e.g, AsCas12a, LbCas12a), Cas12b (e.g., AaCas12b, BhCas12b, BhCas12b V4), Cas12c1, Cas12c2, Cas12 h1, Cas12i1, CasX, CasY, and CasΦ.

A7. The genome editing system of any one of A1-A6, wherein the RNA-guided nuclease is a Cas12a protein.

A8. The genome editing system of any one of A1-A7, wherein the Cas12a protein is a modified Cas12a protein.

A9. The genome editing system of any one of A1-A8, wherein the modified Cas12a protein is an activity enhanced Cas12a protein.

A10. The genome editing system of any one of A1-A9, wherein the RNA-guided nuclease comprises an amino acid sequence selected from the group consisting of SEQ ID NOs: 113-121 and SEQ ID NO: 130.

A11. The genome editing system of any one of A1-A10, wherein the RNA-guided nuclease comprises an amino acid sequence set forth in SEQ ID NO: 130 or SEQ ID NO: 117.

A12. The genome editing system of any one of A1-A11, wherein the gRNA molecule further comprises a Cas12a stem loop.

A13. The genome editing system of any one of A1-A12, wherein the gRNA molecule further comprises a nucleotide extension, wherein the nucleotide extension is a 5′ extension, a 3′ extension, or a combination thereof.

A14. The genome editing system of any one of A1-A13, wherein the nucleotide extension comprises one or more RNA bases, one or more DNA bases, or a combination thereof.

A15. The genome editing system of any one of A1-A14, wherein the gRNA molecule contains one or more modified bases.

A16. The genome editing system of any one of A1-A15, wherein the nucleotide extension is a 5′ extension comprising a nucleotide sequence selected from the group consisting of SEQ ID NOs: 1-23.

A17. The genome editing system of any one of A1-A16, wherein the extension is a 5′ extension comprising the nucleotide sequence set forth in SEQ ID NO: 7.

A18. The genome editing system of any one of A1-A17, wherein the gRNA molecule comprises a DNA/RNA oligonucleotide comprising a nucleotide sequence selected from the group consisting of SEQ ID NOs: 53-81.

A19. The genome editing system of any one of A1-A18, wherein the gRNA molecule comprises the nucleotide sequence set forth in SEQ ID NO: 68, SEQ ID NO: 97, or SEQ ID NO: 112.

B1. The presently disclosed subject matter provides a ribonucleoprotein (RNP) complex comprising the genome editing system of any one of A1-A19.

C1. The presently disclosed subject matter provides a vector for delivering the genome editing system of any one of A1-A19, wherein the vector comprises DNA encoding the gRNA molecule and/or RNA-guided nuclease, RNA encoding the gRNA molecule and/or RNA-guided nuclease, or combination thereof.

D1. The presently disclosed subject matter provides a method of editing a PTPN2 gene in a target cell comprising contacting the target cell with the genome editing system of any one of A1-A19, the RNP complex of B1, or the vector of C1.

E1. The presently disclosed subject matter provides a cell comprising the genome editing system of any one of A1-A19, the RNP complex of B1, or the vector of C1.

E2. The cell of E1, wherein the level of a PTPN2 gene product in the cell is reduced relative to a cell lacking the genome editing system of any one of A1-A19, the RNP complex of B1, or the vector of C1.

E3. The cell of any one of E1-E2, wherein the cell comprises an indel in the target sequence of PTPN2.

F1. The presently disclosed subject matter provides a cell comprising one or more genomic edits in a PTPN2 gene, wherein the cell is edited by the method of D1.

F2. The cell of F1, wherein the one or more genome edits comprise an indel in the target sequence of the PTPN2 gene.

F3. The cell of F2, wherein the indel comprises a deletion of all or a portion of the target sequence of the PTPN2 gene.

F4. The cell of F1-F3, wherein the cell is a T cell.

F5. The cell of F4, wherein the T cell is an alpha/beta T cell.

F6. The cell of any one of F1-F4, wherein the cell further comprises a chimeric antigen receptor (CAR).

F7. The cell of F6, wherein the CAR binds a tumor antigen.

G1. The presently disclosed subject matter provides a composition comprising a population of engineered cells comprising an indel at a target sequence of a PTPN2 gene, wherein the target sequence comprises a nucleotide sequence selected from the group consisting of SEQ ID NOs: 24-52.

G2. The composition of G1, wherein the level of a PTPN2 gene product in the population of cells is reduced relative to a population of non-engineered cells.

G3. The composition of G1 or G2, wherein the engineered cell is a T cell.

G4. The composition of G3, wherein the T cell is an alpha/beta T cell.

H1. The presently disclosed subject matter provides a method of treating a disease or disorder comprising administering to a subject in need thereof the genome editing system of any one of A1-A19, the RNP of B1, the vector of C1, the cell of any one of E1-E3, the cell of any one of F1-F7, or the composition of any one of G1-G4.

H2. The method of H1, wherein the disease or disorder is a tumor, cancer, malignancy, neoplasm, or other proliferative disease or disorder.

H3. The method of H1 or H2, wherein the disease or disorder is selected from the group consisting of leukemia, lymphoma, e.g., chronic lymphocytic leukemia (CLL), acute—lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin, melanoma, bone, and brain cancer, ovarian cancer, epithelial cancers, renal cell carcinoma, pancreatic adenocarcinoma, Hodgkin's lymphoma, cervical carcinoma, colorectal cancer, glioblastoma, neuroblastoma, Ewing's sarcoma, medulloblastoma, osteosarcoma, synovial sarcoma, and/or mesothelioma.

I1. The presently disclosed subject matter provides a gRNA molecule comprising a targeting domain that targets a target sequence of a Protein Tyrosine Phosphatase Non-Receptor Type 2 (PTPN2) gene.

I2. The gRNA molecule of I1, wherein the target sequence of a PTPN2 gene is in exon 6 of PTPN2.

I3. The gRNA molecule of I1 or I2, wherein (a) the target sequence of a PTPN2 gene comprises a nucleotide sequence selected from the group consisting of SEQ ID NO: 29; SEQ ID NO:36; SEQ ID NO: 39; and SEQ ID NO: 51; and/or (b) the targeting domain comprises a nucleotide sequence selected from the group consisting of SEQ ID NO: 58; SEQ ID NO: 65; SEQ ID NO: 68; SEQ ID NO: 73.

I4. The gRNA molecule of I1, wherein (a) the target sequence of a PTPN2 gene comprises a nucleotide sequence selected from the group consisting of SEQ ID NOs: 24-52; and/or (b) the targeting domain comprises a nucleotide sequence selected from the group consisting of SEQ ID NOs: 53-81.

I5. The gRNA molecule of any one of I1-I4, wherein (a) the target sequence of a PTPN2 gene comprises the nucleotide sequence set forth in SEQ ID NO: 39; and/or (b) the targeting domain comprises the nucleotide sequence set forth in SEQ ID NO: 68.

### EXAMPLES

The following Examples are merely illustrative and are not intended to limit the scope or content of the invention in any way.

### Example 1: PTPN2-Targeting RNP Complexes

An in-house bioinformatics tool was utilized for identifying guide RNAs (gRNAs) targeting the PTPN2 gene, located on Chromosome 18. Briefly, CALITAS (CRISPR-Cas-aware Aligner for In silico off-Target Search; Fennell et al., 2021) was employed to identify AsCas12a compatible T-rich protospacer-adjacent motif (PAM) sites within the PTPN2 gene. The program identified every 21-nucleotide long target sequence with a corresponding PAM site, which were filtered to include only those within exons 1-8 of the PTPN2 transcript (ENST00000309660.10). Sequences were also chosen based on site uniqueness in the genome with a single exact match and to exclude sequences with 1 base mismatch and gaps. A list of target sites identified is set forth in Table 4 (N=29).

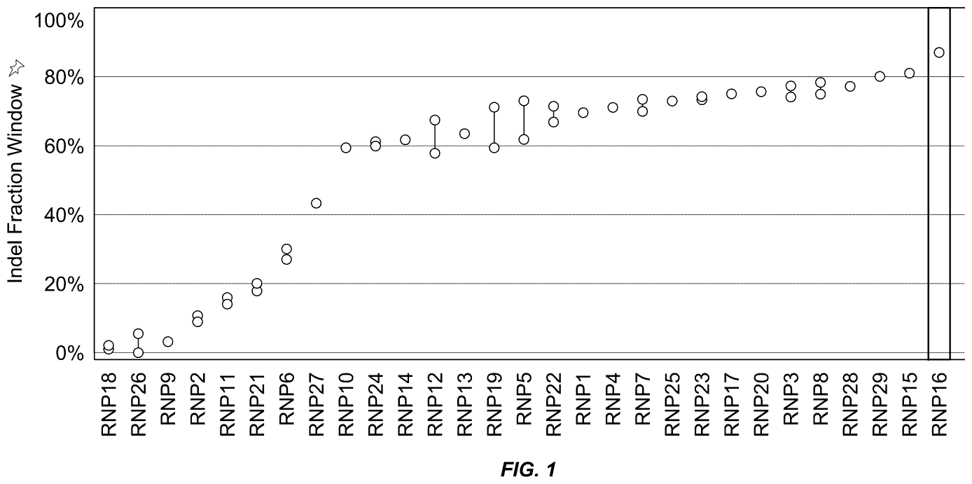

Targeting domains that target the 29 identified target sites were assembled into gRNA oligonucleotides with the addition of an AsCas12a 5′ stem loop. The resulting 41-nucleotide crRNA oligonucleotides were then complexed to an AsCas12a protein (SEQ ID NO: 117). The resulting ribonucleoprotein complexes (RNPs) were formulated at 88 μM with a 2:1 ratio of gRNA to AsCas12a and were transfected into CD4+ T cells by electroporation (Lonza). After incubating for 96 hours, genomic DNA was extracted and evaluated by Next Generation Sequencing (NGS) analysis. Genomic DNA was isolated using the Agencourt DNAdvance kit (Beckman Coulter, Inc.) and quantified using the fluorometric Quant-IT Pico Green dsDNA Assay Kit (ThermoFisher Scientific), each according to the manufacturer's instructions. The editing efficiency for each target sequence was determined by assessing the percentage of sequencing reads that contained indels (editing) that occurred+/−15 bases from the expected cut site for the respective target sequence (FIG. 1). As seen in FIG. 1, RNP16 comprising the crRNA oligonucleotide set forth in [SEQ ID NO: 97] that targets the PTPN2 site set forth in [SEQ ID NO: 39] showed the highest editing efficiency with approximately 90% of sequencing reads containing an indel within the expected cut site window.

The crRNA oligonucleotide set forth in [SEQ ID NO: 97] was joined at the 5′ end by a 25 nucleotide DNA extension set forth in [SEQ ID NO: 7]. The resulting 66 nucleotide DNA/RNA oligonucleotide set forth in [SEQ ID NO: 112] was complexed with an AsCas12a nuclease set forth in [SEQ ID NO: 117] to generate RNP30 shown in Table 14.

RNP30 was functionally validated in a variety of cell types from different subjects and at varying dosages. RNP30 was serially diluted in an equal mixture of buffer 1 (10 mM HEPES pH 7.5, 150 mM NaCl) and buffer 2 (10 mM HEPES pH7.5, 300 mM NaCl, 20% Glycerol, 10 mM TCEP), and CD4+ and CD8+ T cells from multiple donors were electroporated by nucleofection (Lonza) with RNP30 at various concentrations, after which cells were incubated at 37° C. and 5% CO2 for 96 hours. Cell donor and cell types are shown in Table 15.

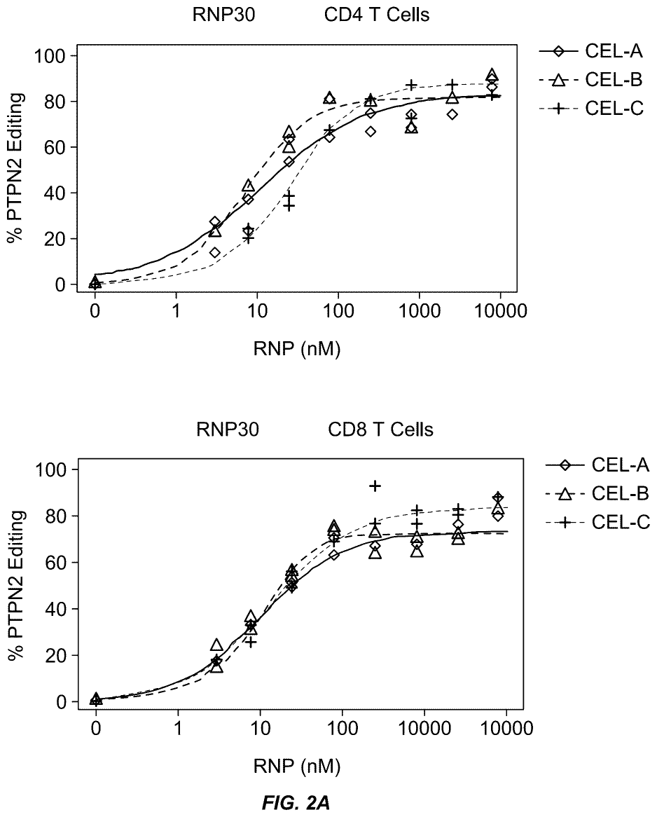

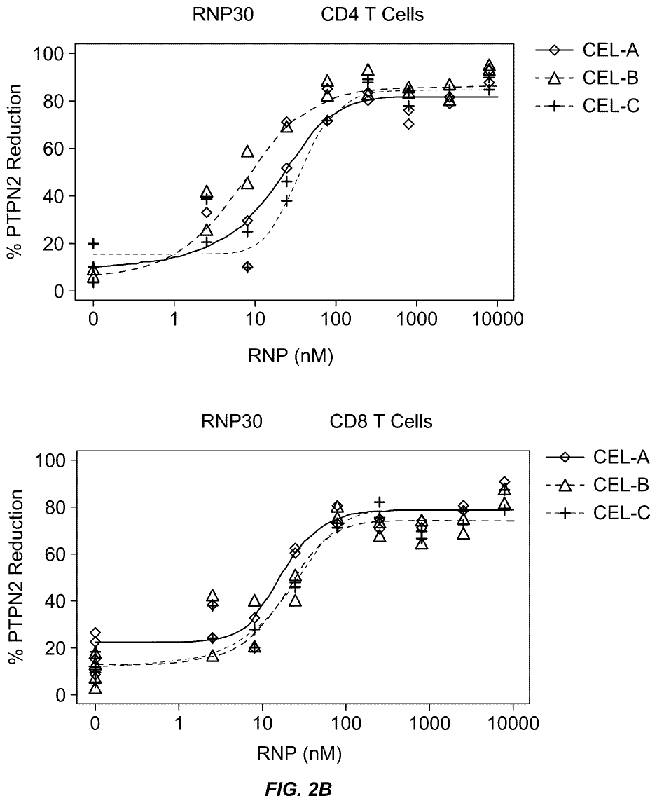

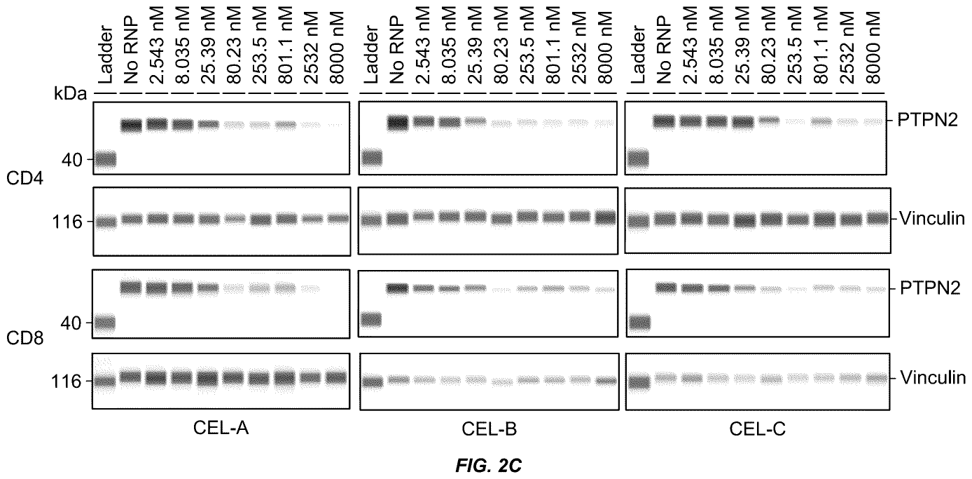

Genomic DNA was extracted as described above 96 hours post incubation and genome editing rates were determined by NGS analysis of a PCR amplicon comprising the expected cut site. A two-step PCR amplification process was used to prepare amplicon libraries for sequencing, with the PCR primers and amplification site shown in Table 8. Editing as measured by Illumina amplicon-based sequencing as a function of concentration is shown in FIG. 2A. The percentage of sequencing reads with an indel+/−15 bases of the expected cut site was used to determine percent editing.

Concentration dependent knockout of PTPN2 at the protein level was assessed 72 hours post nucleofection by an automated Western blot system (JESS, Proteinsimple) that is capillary based and determines protein size and abundance by chemiluminescence. Total protein was quantified using a bicinchoninic acid (BCA) kit (ThermoFisher) in accordance with the manufacturer's instructions. Rabbit anti-PTPN2 (Cell Signaling Technologies Cat #58935S) monoclonal antibody and rabbit anti-Vinculin (Abcam Cat #ab155120) monoclonal loading control antibody were used to quantify PTPN2 protein expression. PTPN2 protein levels were determined using the ratio of the PTPN2 protein chemiluminescent signal peaks normalized to the Vinculin non-targeted control protein signal peak. Rendered blot representations of protein abundance are shown in FIG. 2C and PTPN2 protein reduction as a function of RNP concentration is shown in FIG. 2B.

EC50 values for percent editing and protein inhibition are shown in Table 15. Concentration response curves shown in FIGS. 2A-B were fit with a four-parametric logistic regression equation. The geometric average EC50 for editing was 12.26 nM and the geometric average EC50 for PTPN2 knockdown was 18.29 nM. These results demonstrate that the editing activity of RNP30 increases in a concentration dependent manner and that the editing results in reduced PTPN2 protein levels. Furthermore, this response is consistent across CD4+ and CD8+ T cells from the same donor as well as from three different donors. Thus, RNP30 which targets PTPN2 at the site set forth in [SEQ ID NO: 39] can edit and knock-out the PTPN2 gene in human cells.

TABLE 15
Cell Lines in RNP30 Characterization with Corresponding EC50s

| | Cell | % Editing | % PTPN2 Knockdown |
|---|---|---|---|
| Cell ID | Subset | EC50 (nM) | EC50 (nM) |
| CEL-A-001 | CD4 | 11.19 | 19.35 |
| CEL-B-001 | CD4 | 7.48 | 7.41 |
| CEL-C-001 | CD4 | 26.99 | 36.43 |
| CEL-A-002 | CD4 | 10.57 | 16.68 |
| CEL-B-002 | CD8 | 10.32 | 19.17 |
| CEL-C-002 | CD8 | 13.78 | 22.39 |

| | | |
|---|---|---|
| Geometric Mean | 12.26 | 18.29 |

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

1. A genome editing system comprising:
(a) a gRNA molecule comprising a targeting domain that targets a target sequence of a Protein Tyrosine Phosphatase Non-Receptor Type 2 (PTPN2) gene, and
(b) an RNA-guided nuclease, or a nucleic acid encoding the RNA-guided nuclease.

2. The genome editing system of claim 1, wherein the target sequence of a PTPN2 gene is in exon 6 of PTPN2.

3. The genome editing system of claim 1, wherein
(a) the target sequence of a PTPN2 gene comprises a nucleotide sequence selected from the group consisting of SEQ ID NOs: 24-52; and/or
(b) the targeting domain comprises a nucleotide sequence selected from the group consisting of SEQ ID NOs: 53-81.

4. (canceled)

5. The genome editing system of claim 1, wherein
(a) the target sequence of a PTPN2 gene comprises the nucleotide sequence set forth in SEQ ID NO: 39; and/or
(b) the targeting domain comprises the nucleotide sequence set forth in SEQ ID NO: 68.

6. The genome editing system of claim 1, wherein the RNA-guided nuclease is selected from the group consisting of Cas9 (e.g., SpCas9, SaCas9, (KKH) SaCas9, eSpCas9, Cas9-HF1, HypaCas9, dCas9-Fokl, Sniper-Cas9, xCas9, evoCas9, SpCas9-NG, VRQR, VRER, NmeCas9, CjCas9), Cas12, Cas12a (also known as Cpf1; e.g, AsCas12a, LbCas12a), Cas12b (e.g., AaCas12b, BhCas12b, BhCas12b V4), Cas12c1, Cas12c2, Cas12h1, Cas12i1, CasX, CasY, and CasΦ.

7. The genome editing system of claim 1, wherein the RNA-guided nuclease is a Cas12a protein, a modified Cas12a protein, and/or an activity enhanced Cas12a protein.

8.-9. (canceled)

10. The genome editing system of claim 1, wherein the RNA-guided nuclease comprises an amino acid sequence selected from the group consisting of SEQ ID NOs: 113-121 and SEQ ID NO: 130.

11. (canceled)

12. The genome editing system of claim 1, wherein the gRNA molecule further comprises:
a) a Cas12a stem loop,
b) a nucleotide extension, wherein the nucleotide extension is a 5′extension, a 3′ extension, or a combination thereof; and/or
c) one or more modified bases.

13-15. (canceled)

16. The genome editing system of claim 12, wherein the nucleotide extension is a 5′ extension comprising a nucleotide sequence selected from the group consisting of SEQ ID NOs: 1-23.

17. (canceled)

18. The genome editing system of claim 1, wherein the gRNA molecule comprises a DNA/RNA oligonucleotide comprising a nucleotide sequence selected from the group consisting of SEQ ID NOs: 53-81.

19. The genome editing system of claim 1, wherein the gRNA molecule comprises the nucleotide sequence set forth in SEQ ID NO: 68, SEQ ID NO: 97, or SEQ ID NO: 112.

20. A ribonucleoprotein (RNP) complex comprising the genome editing system of claim 1.

21. A vector for delivering the genome editing system of claim 1, wherein the vector comprises DNA encoding the gRNA molecule and/or RNA-guided nuclease, RNA encoding the gRNA molecule and/or RNA-guided nuclease, or combination thereof.

22. A method of editing a PTPN2 gene in a target cell comprising contacting the target cell with the genome editing system of claim 1.

23. A cell comprising the genome editing system of claim 1.

24.-25. (canceled)

26. A cell comprising one or more genomic edits in a PTPN2 gene, wherein the cell is edited by the method of claim 22.

27.-32. (canceled)

33. A composition comprising a population of engineered cells comprising an indel at a target sequence of a PTPN2 gene, wherein the target sequence comprises a nucleotide sequence selected from the group consisting of SEQ ID NOs: 24-52.

34.-36. (canceled)

37. A method of treating a disease or disorder comprising administering to a subject in need thereof the genome editing system of claim 1 or a cell comprising one or more genomic edits in a PTPN2 gene generated by the genome editing system.

38.-39. (canceled)

40. A gRNA molecule comprising a targeting domain that targets a target sequence of a Protein Tyrosine Phosphatase Non-Receptor Type 2 (PTPN2) gene.

41. (canceled)

42. The gRNA molecule of claim 40, wherein
(a) the target sequence of a PTPN2 gene comprises a nucleotide sequence selected from the group consisting of SEQ ID NO: 29; SEQ ID NO:36; SEQ ID NO: 39; and SEQ ID NO: 51; and/or
(b) the targeting domain comprises a nucleotide sequence selected from the group consisting of SEQ ID NO: 58; SEQ ID NO: 65; SEQ ID NO: 68; SEQ ID NO: 73.

43.-44. (canceled)


SEQUENCE LISTING (131 sequences)

SEQ ID NO: 1 - skipped

SEQ ID NO: 2 - RNA, 10 nt, synthetic construct
  misc_feature 1..10: note = Description of sequence: 5 extension sequence, 5 modification: +5 RNA
  source 1..10: mol_type = other RNA
aagacctttt

SEQ ID NO: 3 - RNA, 25 nt, synthetic construct
  misc_feature 1..25: note = Description of sequence: 5 extension sequence, 5 modification: +10 RNA
  source 1..25: mol_type = other RNA
atgtgtttttgtcaaaagacctttt

SEQ ID NO: 4 - RNA, 60 nt, synthetic construct
  misc_feature 1..60: note = Description of sequence: 5 extension sequence, 5 modification: +25 RNA
  source 1..60: mol_type = other RNA
aggccagcttgccggttttttagtcgtgctgcttcatgtgtttttgtcaaaagacctttt

SEQ ID NO: 5 - skipped

SEQ ID NO: 6 - RNA, 10 nt, synthetic construct
  misc_feature 1..10: note = Description of sequence: 5 extension sequence, 5 modification: +5 DNA
  source 1..10: mol_type = other RNA
aagacctttt

SEQ ID NO: 7 - RNA, 25 nt, synthetic construct
  misc_feature 1..25: note = Description of sequence: 5 extension sequence, 5 modification: +10 DNA
  source 1..25: mol_type = other RNA
atgtgtttttgtcaaaagacctttt

SEQ ID NO: 8 - RNA, 40 nt, synthetic construct
  misc_feature 1..40: note = Description of sequence: 5 extension sequence, 5 modification: +25 DNA
  source 1..40: mol_type = other RNA
aggccagcttgccggttttttagtcgtgctgcttcatgtg

SEQ ID NO: 9 - RNA, 20 nt, synthetic construct
  misc_feature 1..20: note = Description of sequence: 5 extension sequence, 5 modification: +60 DNA
  source 1..20: mol_type = other RNA
tttttgtcaaaagacctttt

SEQ ID NO: 10 - RNA, 30 nt, synthetic construct
  misc_feature 1..30: note = Description of sequence: 5 extension sequence, 5 modification: +20 DNA
  source 1..30: mol_type = other RNA
gcttcatgtgtttttgtcaaaagacctttt

SEQ ID NO: 11 - RNA, 50 nt, synthetic construct
  misc_feature 1..50: note = Description of sequence: 5 extension sequence, 5 modification: +30 DNA
  source 1..50: mol_type = other RNA
gccggttttttagtcgtgctgcttcatgtgtttttgtcaaaagacctttt

SEQ ID NO: 12 - RNA, 40 nt, synthetic construct
  misc_feature 1..40: note = Description of sequence: 5 extension sequence, 5 modification: +50 DNA
  source 1..40: mol_type = other RNA
tagtcgtgctgcttcatgtgtttttgtcaaaagacctttt

SEQ ID NO: 13 - RNA, 20 nt, synthetic construct
  misc_feature 1..20: note = Description of sequence: 5 extension sequence, 5 modification: +40 DNA
  source 1..20: mol_type = other RNA
  modified_base 1^2: mod_base = OTHER; note = A phosphorothioate llinkage is present between these bases
  modified_base 2^3: mod_base = OTHER; note = A phosphorothioate llinkage is present between these bases
ccgaagttttcttcggtttt

SEQ ID NO: 14 - RNA, 25 nt, synthetic construct
  misc_feature 1..25: note = Description of sequence: 5 extension sequence, 5 modification: +20 DNA + 2xPS
  source 1..25: mol_type = other RNA
  modified_base 1^2: mod_base = OTHER; note = A phosphorothioate llinkage is present between these bases
  modified_base 2^3: mod_base = OTHER; note = A phosphorothioate llinkage is present between these bases
tttttccgaagttttcttcggtttt

SEQ ID NO: 15 - RNA, 30 nt, synthetic construct
  misc_feature 1..30: note = Description of sequence: 5 extension sequence, 5 modification: +25 DNA + 2xPS
  source 1..30: mol_type = other RNA
  modified_base 1^2: mod_base = OTHER; note = A phosphorothioate llinkage is present between these bases
  modified_base 2^3: mod_base = OTHER; note = A phosphorothioate llinkage is present between these bases
aacgctttttccgaagttttcttcggtttt

SEQ ID NO: 16 - RNA, 41 nt, synthetic construct
  misc_feature 1..41: note = Description of sequence: 5 extension sequence, 5 modification: +30 DNA + 2xPS
  source 1..41: mol_type = other RNA
  modified_base 2^3: mod_base = OTHER; note = A phosphorothioate llinkage is present between these bases
  modified_base 2^3: mod_base = OTHER; note = A phosphorothioate llinkage is present between these bases
gcgttgttttcaacgctttttccgaagttttcttcggtttt

SEQ ID NO: 17 - RNA, 62 nt, synthetic construct
  misc_feature 1..62: note = Description of sequence: 5 extension sequence, 5 modification: +41 DNA + 2xPS
  source 1..62: mol_type = other RNA
  modified_base 1^2: mod_base = OTHER; note = A phosphorothioate llinkage is present between these bases
  modified_base 2^3: mod_base = OTHER; note = A phosphorothioate llinkage is present between these bases
ggcttcttttgaagcctttttgcgttgttttcaacgctttttccgaagttttcttcggtttt

SEQ ID NO: 18 - RNA, 25 nt, synthetic construct
  misc_feature 1..25: note = Description of sequence: 5 extension sequence, 5 modification: +62 DNA + 2xPS
  source 1..25: mol_type = other RNA
  modified_base 1^2: mod_base = OTHER; note = A phosphorothioate llinkage is present between these bases
  modified_base 2^3: mod_base = OTHER; note = A phosphorothioate llinkage is present between these bases
atgtgtttttgtcaaaagacctttt

SEQ ID NO: 19 - RNA, 25 nt, synthetic construct
  misc_feature 1..25: note = Description of sequence: 5 extension sequence, 5 modification: +25 DNA + 2xPS
  source 1..25: mol_type = other RNA
aaaaaaaaaaaaaaaaaaaaaaaaa

SEQ ID NO: 20 - RNA, 25 nt, synthetic construct
  misc_feature 1..25: note = Description of sequence: 5 extension sequence, 5 modification: +25 A
  source 1..25: mol_type = other RNA
ttttttttttttttttttttttttt

SEQ ID NO: 21 - RNA, 25 nt, synthetic construct
  misc_feature 1..25: note = Description of sequence: 5 extension sequence, 5 modification: +25 T
  source 1..25: mol_type = other RNA
  modified_base 1^2: mod_base = OTHER; note = A phosphorothioate llinkage is present between these bases
  modified_base 2^3: mod_base = OTHER; note = A phosphorothioate llinkage is present between these bases
  modified_base 1: mod_base = OTHER; note = 2'-O-methyl-adenosine
  modified_base 2: mod_base = um
atgtgtttttgtcaaaagacctttt

SEQ ID NO: 22 - RNA, 25 nt, synthetic construct
  misc_feature 1..25: note = Description of sequence: 5 extension sequence, 5 modification: +25 RNA + 2xPS
  source 1..25: mol_type = other RNA
  modified_base 1^2: mod_base = OTHER; note = A phosphorothioate llinkage is present between these bases
  modified_base 2^3: mod_base = OTHER; note = A phosphorothioate llinkage is present between these bases
  modified_base 1: mod_base = OTHER; note = 2'-O-methyl-adenosine
  modified_base 2: mod_base = OTHER; note = 2'-O-methyl-adenosine
aaaaaaaaaaaaaaaaaaaaaaaaa

SEQ ID NO: 23 - RNA, 25 nt, synthetic construct
  misc_feature 1..25: note = Description of sequence: 5 extension sequence, 5 modification: PolyA RNA + 2xPS
  source 1..25: mol_type = other RNA
  modified_base 1^2: mod_base = OTHER; note = A phosphorothioate llinkage is present between these bases
  modified_base 2^3: mod_base = OTHER; note = A phosphorothioate llinkage is present between these bases
  modified_base 1: mod_base = um
  modified_base 2: mod_base = um
ttttttttttttttttttttttttt

SEQ ID NO: 24 - DNA, 21 bp, synthetic construct
  misc_feature 1..21: note = Description of sequence: gRNA Target Sequence (DNA), RNP ID: RNP1, Target Site ID: RefSeq1
  source 1..21: mol_type = other DNA
taaggaagacttatctcctgc

SEQ ID NO: 25 - DNA, 21 bp, synthetic construct
  misc_feature 1..21: note = Description of sequence: gRNA Target Sequence (DNA), RNP ID: RNP2, Target Site ID: RefSeq2
  source 1..21: mol_type = other DNA
tgcctttcttgcagatggaaa

SEQ ID NO: 26 - DNA, 21 bp, synthetic construct
  misc_feature 1..21: note = Description of sequence: gRNA Target Sequence (DNA), RNP ID: RNP3, Target Site ID: RefSeq3
  source 1..21: mol_type = other DNA
ggttaaatgtgcacagtactg

SEQ ID NO: 27 - DNA, 21 bp, synthetic construct
  misc_feature 1..21: note = Description of sequence: gRNA Target Sequence (DNA), RNP ID: RNP4, Target Site ID: RefSeq4
  source 1..21: mol_type = other DNA
ttattttaaggaaattcgaaa

SEQ ID NO: 28 - DNA, 21 bp, synthetic construct
  misc_feature 1..21: note = Description of sequence: gRNA Target Sequence (DNA), RNP ID: RNP5, Target Site ID: RefSeq5
  source 1..21: mol_type = other DNA
cctgtatactagaatctccct

SEQ ID NO: 29 - DNA, 21 bp, synthetic construct
  misc_feature 1..21: note = Description of sequence: gRNA Target Sequence (DNA), RNP ID: RNP6, Target Site ID: RefSeq6
  source 1..21: mol_type = other DNA
tagagtggtgaaaccagaaca

SEQ ID NO: 30 - DNA, 21 bp, synthetic construct
  misc_feature 1..21: note = Description of sequence: gRNA Target Sequence (DNA), RNP ID: RNP7, Target Site ID: RefSeq7
  source 1..21: mol_type = other DNA
cagatgatcacagtcgtgtta

SEQ ID NO: 31 - DNA, 21 bp, synthetic construct
  misc_feature 1..21: note = Description of sequence: gRNA Target Sequence (DNA), RNP ID: RNP8, Target Site ID: RefSeq8
  source 1..21: mol_type = other DNA
tcttccctacctagaaacgat

SEQ ID NO: 32 - DNA, 21 bp, synthetic construct
  misc_feature 1..21: note = Description of sequence: gRNA Target Sequence (DNA), RNP ID: RNP9, Target Site ID: RefSeq9
  source 1..21: mol_type = other DNA
ttgcctttcttgcagatggaa

SEQ ID NO: 33 - DNA, 21 bp, synthetic construct
  misc_feature 1..21: note = Description of sequence: gRNA Target Sequence (DNA), RNP ID: RNP10, Target Site ID: RefSeq10
  source 1..21: mol_type = other DNA
taaatttaggttaaatgtgca

SEQ ID NO: 34 - DNA, 21 bp, synthetic construct
  misc_feature 1..21: note = Description of sequence: gRNA Target Sequence (DNA), RNP ID: RNP11, Target Site ID: RefSeq11
  source 1..21: mol_type = other DNA
gttattttaaggaaattcgaa

SEQ ID NO: 35 - DNA, 21 bp, synthetic construct
  misc_feature 1..21: note = Description of sequence: gRNA Target Sequence (DNA), RNP ID: RNP12, Target Site ID: RefSeq12
  source 1..21: mol_type = other DNA
ttgcagatggaaaaaggagat

SEQ ID NO: 36 - DNA, 21 bp, synthetic construct
  misc_feature 1..21: note = Description of sequence: gRNA Target Sequence (DNA), RNP ID: RNP13, Target Site ID: RefSeq13
  source 1..21: mol_type = other DNA
actttttagagtggtgaaacc

SEQ ID NO: 37 - DNA, 21 bp, synthetic construct
  misc_feature 1..21: note = Description of sequence: gRNA Target Sequence (DNA), RNP ID: RNP14, Target Site ID: RefSeq14
  source 1..21: mol_type = other DNA
tcattctctattttcagatga

SEQ ID NO: 38 - DNA, 21 bp, synthetic construct
  misc_feature 1..21: note = Description of sequence: gRNA Target Sequence (DNA), RNP ID: RNP15, Target Site ID: RefSeq15
  source 1..21: mol_type = other DNA
atttctcttccctacctagaa

SEQ ID NO: 39 - DNA, 21 bp, synthetic construct
  misc_feature 1..21: note = Description of sequence: gRNA Target Sequence (DNA), RNP ID: RNP16, Target Site ID: RefSeq16
  source 1..21: mol_type = other DNA
gagtggtgaaaccagaacaat

SEQ ID NO: 40 - DNA, 21 bp, synthetic construct
  misc_feature 1..21: note = Description of sequence: gRNA Target Sequence (DNA), RNP ID: RNP17, Target Site ID: RefSeq17
  source 1..21: mol_type = other DNA
gttgacatagaagaggcacaa

SEQ ID NO: 41 - DNA, 21 bp, synthetic construct
  misc_feature 1..21: note = Description of sequence: gRNA Target Sequence (DNA), RNP ID: RNP18, Target Site ID: RefSeq18
  source 1..21: mol_type = other DNA
tgttattttaaggaaattcga

SEQ ID NO: 42 - DNA, 21 bp, synthetic construct
  misc_feature 1..21: note = Description of sequence: gRNA Target Sequence (DNA), RNP ID: RNP19, Target Site ID: RefSeq19
  source 1..21: mol_type = other DNA
agaggaaagtcctgtacatcg

SEQ ID NO: 43 - DNA, 21 bp, synthetic construct
  misc_feature 1..21: note = Description of sequence: gRNA Target Sequence (DNA), RNP ID: RNP20, Target Site ID: RefSeq20
  source 1..21: mol_type = other DNA
cctttcttgcagatggaaaaa

SEQ ID NO: 44 - DNA, 21 bp, synthetic construct
  misc_feature 1..21: note = Description of sequence: gRNA Target Sequence (DNA), RNP ID: RNP21, Target Site ID: RefSeq21
  source 1..21: mol_type = other DNA
cactttttagagtggtgaaac

SEQ ID NO: 45 - DNA, 21 bp, synthetic construct
  misc_feature 1..21: note = Description of sequence: gRNA Target Sequence (DNA), RNP ID: RNP22, Target Site ID: RefSeq22
  source 1..21: mol_type = other DNA
aggaaattcgaaatgagtccc

SEQ ID NO: 46 - DNA, 21 bp, synthetic construct
  misc_feature 1..21: note = Description of sequence: gRNA Target Sequence (DNA), RNP ID: RNP23, Target Site ID: RefSeq23
  source 1..21: mol_type = other DNA
gaggaaagtcctgtacatcgg

SEQ ID NO: 47 - DNA, 21 bp, synthetic construct
  misc_feature 1..21: note = Description of sequence: gRNA Target Sequence (DNA), RNP ID: RNP24, Target Site ID: RefSeq24
  source 1..21: mol_type = other DNA
gcctttcttgcagatggaaaa

SEQ ID NO: 48 - DNA, 21 bp, synthetic construct
  misc_feature 1..21: note = Description of sequence: gRNA Target Sequence (DNA), RNP ID: RNP25, Target Site ID: RefSeq25
  source 1..21: mol_type = other DNA
gggcaagaaaggtcttacatt

SEQ ID NO: 49 - DNA, 21 bp, synthetic construct
  misc_feature 1..21: note = Description of sequence: gRNA Target Sequence (DNA), RNP ID: RNP26, Target Site ID: RefSeq26
  source 1..21: mol_type = other DNA
aaggaaattcgaaatgagtcc

SEQ ID NO: 50 - DNA, 21 bp, synthetic construct
  misc_feature 1..21: note = Description of sequence: gRNA Target Sequence (DNA), RNP ID: RNP27, Target Site ID: RefSeq27
  source 1..21: mol_type = other DNA
aaacctgtatgaagtttacct

SEQ ID NO: 51 - DNA, 21 bp, synthetic construct
  misc_feature 1..21: note = Description of sequence: gRNA Target Sequence (DNA), RNP ID: RNP28, Target Site ID: RefSeq28
  source 1..21: mol_type = other DNA
agagtggtgaaaccagaacaa

SEQ ID NO: 52 - DNA, 21 bp, synthetic construct
  misc_feature 1..21: note = Description of sequence: gRNA Target Sequence (DNA), RNP ID: RNP29, Target Site ID: RefSeq29
  source 1..21: mol_type = other DNA
agatgatcacagtcgtgttaa

SEQ ID NO: 53 - RNA, 21 nt, synthetic construct
  misc_feature 1..21: note = Description of sequence: gRNA Target Sequence (RNA), Target Site ID: RefSeq1
  source 1..21: mol_type = other RNA
taaggaagacttatctcctgc

SEQ ID NO: 54 - RNA, 21 nt, synthetic construct
  misc_feature 1..21: note = Description of sequence: gRNA Target Sequence (RNA), Target Site ID: RefSeq2
  source 1..21: mol_type = other RNA
tgcctttcttgcagatggaaa

SEQ ID NO: 55 - RNA, 21 nt, synthetic construct
  misc_feature 1..21: note = Description of sequence: gRNA Target Sequence (RNA), Target Site ID: RefSeq3
  source 1..21: mol_type = other RNA
ggttaaatgtgcacagtactg

SEQ ID NO: 56 - RNA, 21 nt, synthetic construct
  misc_feature 1..21: note = Description of sequence: gRNA Target Sequence (RNA), Target Site ID: RefSeq4
  source 1..21: mol_type = other RNA
ttattttaaggaaattcgaaa

SEQ ID NO: 57 - RNA, 21 nt, synthetic construct
  misc_feature 1..21: note = Description of sequence: gRNA Target Sequence (RNA), Target Site ID: RefSeq5
  source 1..21: mol_type = other RNA
cctgtatactagaatctccct

SEQ ID NO: 58 - RNA, 21 nt, synthetic construct
  misc_feature 1..21: note = Description of sequence: gRNA Target Sequence (RNA), Target Site ID: RefSeq6
  source 1..21: mol_type = other RNA
tagagtggtgaaaccagaaca

SEQ ID NO: 59 - RNA, 21 nt, synthetic construct
  misc_feature 1..21: note = Description of sequence: gRNA Target Sequence (RNA), Target Site ID: RefSeq7
  source 1..21: mol_type = other RNA
cagatgatcacagtcgtgtta

SEQ ID NO: 60 - RNA, 21 nt, synthetic construct
  misc_feature 1..21: note = Description of sequence: gRNA Target Sequence (RNA), Target Site ID: RefSeq8
  source 1..21: mol_type = other RNA
tcttccctacctagaaacgat

SEQ ID NO: 61 - RNA, 21 nt, synthetic construct
  misc_feature 1..21: note = Description of sequence: gRNA Target Sequence (RNA), Target Site ID: RefSeq9
  source 1..21: mol_type = other RNA
ttgcctttcttgcagatggaa

SEQ ID NO: 62 - RNA, 21 nt, synthetic construct
  misc_feature 1..21: note = Description of sequence: gRNA Target Sequence (RNA), Target Site ID: RefSeq10
  source 1..21: mol_type = other RNA
taaatttaggttaaatgtgca

SEQ ID NO: 63 - RNA, 21 nt, synthetic construct
  misc_feature 1..21: note = Description of sequence: gRNA Target Sequence (RNA), Target Site ID: RefSeq11
  source 1..21: mol_type = other RNA
gttattttaaggaaattcgaa

SEQ ID NO: 64 - RNA, 21 nt, synthetic construct
  misc_feature 1..21: note = Description of sequence: gRNA Target Sequence (RNA), Target Site ID: RefSeq12
  source 1..21: mol_type = other RNA
ttgcagatggaaaaaggagat

SEQ ID NO: 65 - RNA, 21 nt, synthetic construct
  misc_feature 1..21: note = Description of sequence: gRNA Target Sequence (RNA), Target Site ID: RefSeq13
  source 1..21: mol_type = other RNA
actttttagagtggtgaaacc

SEQ ID NO: 66 - RNA, 21 nt, synthetic construct
  misc_feature 1..21: note = Description of sequence: gRNA Target Sequence (RNA), Target Site ID: RefSeq14
  source 1..21: mol_type = other RNA
tcattctctattttcagatga

SEQ ID NO: 67 - RNA, 21 nt, synthetic construct
  misc_feature 1..21: note = Description of sequence: gRNA Target Sequence (RNA), Target Site ID: RefSeq15
  source 1..21: mol_type = other RNA
atttctcttccctacctagaa

SEQ ID NO: 68 - RNA, 21 nt, synthetic construct
  misc_feature 1..21: note = Description of sequence: gRNA Target Sequence (RNA), Target Site ID: RefSeq16
  source 1..21: mol_type = other RNA
gagtggtgaaaccagaacaat

SEQ ID NO: 69 - RNA, 21 nt, synthetic construct
  misc_feature 1..21: note = Description of sequence: gRNA Target Sequence (RNA), Target Site ID: RefSeq17
  source 1..21: mol_type = other RNA
gttgacatagaagaggcacaa

SEQ ID NO: 70 - RNA, 21 nt, synthetic construct
  misc_feature 1..21: note = Description of sequence: gRNA Target Sequence (RNA), Target Site ID: RefSeq18
  source 1..21: mol_type = other RNA
tgttattttaaggaaattcga

SEQ ID NO: 71 - RNA, 21 nt, synthetic construct
  misc_feature 1..21: note = Description of sequence: gRNA Target Sequence (RNA), Target Site ID: RefSeq19
  source 1..21: mol_type = other RNA
agaggaaagtcctgtacatcg

SEQ ID NO: 72 - RNA, 21 nt, synthetic construct
  misc_feature 1..21: note = Description of sequence: gRNA Target Sequence (RNA), Target Site ID: RefSeq20
  source 1..21: mol_type = other RNA
cctttcttgcagatggaaaaa

SEQ ID NO: 73 - RNA, 21 nt, synthetic construct
  misc_feature 1..21: note = Description of sequence: gRNA Target Sequence (RNA), Target Site ID: RefSeq21
  source 1..21: mol_type = other RNA
cactttttagagtggtgaaac

SEQ ID NO: 74 - RNA, 21 nt, synthetic construct
  misc_feature 1..21: note = Description of sequence: gRNA Target Sequence (RNA), Target Site ID: RefSeq22
  source 1..21: mol_type = other RNA
aggaaattcgaaatgagtccc

SEQ ID NO: 75 - RNA, 21 nt, synthetic construct
  misc_feature 1..21: note = Description of sequence: gRNA Target Sequence (RNA), Target Site ID: RefSeq23
  source 1..21: mol_type = other RNA
gaggaaagtcctgtacatcgg

SEQ ID NO: 76 - RNA, 21 nt, synthetic construct
  misc_feature 1..21: note = Description of sequence: gRNA Target Sequence (RNA), Target Site ID: RefSeq24
  source 1..21: mol_type = other RNA
gcctttcttgcagatggaaaa

SEQ ID NO: 77 - RNA, 21 nt, synthetic construct
  misc_feature 1..21: note = Description of sequence: gRNA Target Sequence (RNA), Target Site ID: RefSeq25
  source 1..21: mol_type = other RNA
gggcaagaaaggtcttacatt

SEQ ID NO: 78 - RNA, 21 nt, synthetic construct
  misc_feature 1..21: note = Description of sequence: gRNA Target Sequence (RNA), Target Site ID: RefSeq26
  source 1..21: mol_type = other RNA
aaggaaattcgaaatgagtcc

SEQ ID NO: 79 - RNA, 21 nt, synthetic construct
  misc_feature 1..21: note = Description of sequence: gRNA Target Sequence (RNA), Target Site ID: RefSeq27
  source 1..21: mol_type = other RNA
aaacctgtatgaagtttacct

SEQ ID NO: 80 - RNA, 21 nt, synthetic construct
  misc_feature 1..21: note = Description of sequence: gRNA Target Sequence (RNA), Target Site ID: RefSeq28
  source 1..21: mol_type = other RNA
agagtggtgaaaccagaacaa

SEQ ID NO: 81 - RNA, 21 nt, synthetic construct
  misc_feature 1..21: note = Description of sequence: gRNA Target Sequence (RNA), Target Site ID: RefSeq29
  source 1..21: mol_type = other RNA
agatgatcacagtcgtgttaa

SEQ ID NO: 82 - DNA, 41 bp, synthetic construct
  misc_feature 1..41: note = Description of sequence: crRNA, RNP ID: RNP1, Target Site ID: RefSeq1
  source 1..41: mol_type = other DNA
taatttctactcttgtagattaaggaagacttatctcctgc

SEQ ID NO: 83 - DNA, 41 bp, synthetic construct
  misc_feature 1..41: note = Description of sequence: crRNA, RNP ID: RNP2, Target Site ID: RefSeq2
  source 1..41: mol_type = other DNA
taatttctactcttgtagattgcctttcttgcagatggaaa

SEQ ID NO: 84 - DNA, 41 bp, synthetic construct
  misc_feature 1..41: note = Description of sequence: crRNA, RNP ID: RNP3, Target Site ID: RefSeq3
  source 1..41: mol_type = other DNA
taatttctactcttgtagatggttaaatgtgcacagtactg

SEQ ID NO: 85 - DNA, 41 bp, synthetic construct
  misc_feature 1..41: note = Description of sequence: crRNA, RNP ID: RNP4, Target Site ID: RefSeq4
  source 1..41: mol_type = other DNA
taatttctactcttgtagatttattttaaggaaattcgaaa

SEQ ID NO: 86 - DNA, 41 bp, synthetic construct
  misc_feature 1..41: note = Description of sequence: crRNA, RNP ID: RNP5, Target Site ID: RefSeq5
  source 1..41: mol_type = other DNA
taatttctactcttgtagatcctgtatactagaatctccct

SEQ ID NO: 87 - DNA, 41 bp, synthetic construct
  misc_feature 1..41: note = Description of sequence: crRNA, RNP ID: RNP6, Target Site ID: RefSeq6
  source 1..41: mol_type = other DNA
taatttctactcttgtagattagagtggtgaaaccagaaca

SEQ ID NO: 88 - DNA, 41 bp, synthetic construct
  misc_feature 1..41: note = Description of sequence: crRNA, RNP ID: RNP7, Target Site ID: RefSeq7
  source 1..41: mol_type = other DNA
taatttctactcttgtagatcagatgatcacagtcgtgtta

SEQ ID NO: 89 - DNA, 41 bp, synthetic construct
  misc_feature 1..41: note = Description of sequence: crRNA, RNP ID: RNP8, Target Site ID: RefSeq8
  source 1..41: mol_type = other DNA
taatttctactcttgtagattcttccctacctagaaacgat

SEQ ID NO: 90 - DNA, 41 bp, synthetic construct
  misc_feature 1..41: note = Description of sequence: crRNA, RNP ID: RNP9, Target Site ID: RefSeq9
  source 1..41: mol_type = other DNA
taatttctactcttgtagatttgcctttcttgcagatggaa

SEQ ID NO: 91 - DNA, 41 bp, synthetic construct
  misc_feature 1..41: note = Description of sequence: crRNA, RNP ID: RNP10, Target Site ID: RefSeq10
  source 1..41: mol_type = other DNA
taatttctactcttgtagattaaatttaggttaaatgtgca

SEQ ID NO: 92 - DNA, 41 bp, synthetic construct
  misc_feature 1..41: note = Description of sequence: crRNA, RNP ID: RNP11, Target Site ID: RefSeq11
  source 1..41: mol_type = other DNA
taatttctactcttgtagatgttattttaaggaaattcgaa

SEQ ID NO: 93 - DNA, 41 bp, synthetic construct
  misc_feature 1..41: note = Description of sequence: crRNA, RNP ID: RNP12, Target Site ID: RefSeq12
  source 1..41: mol_type = other DNA
taatttctactcttgtagatttgcagatggaaaaaggagat

SEQ ID NO: 94 - DNA, 41 bp, synthetic construct
  misc_feature 1..41: note = Description of sequence: crRNA, RNP ID: RNP13, Target Site ID: RefSeq13
  source 1..41: mol_type = other DNA
taatttctactcttgtagatactttttagagtggtgaaacc

SEQ ID NO: 95 - DNA, 41 bp, synthetic construct
  misc_feature 1..41: note = Description of sequence: crRNA, RNP ID: RNP14, Target Site ID: RefSeq14
  source 1..41: mol_type = other DNA
taatttctactcttgtagattcattctctattttcagatga

SEQ ID NO: 96 - DNA, 41 bp, synthetic construct
  misc_feature 1..41: note = Description of sequence: crRNA, RNP ID: RNP15, Target Site ID: RefSeq15
  source 1..41: mol_type = other DNA
taatttctactcttgtagatatttctcttccctacctagaa

SEQ ID NO: 97 - DNA, 41 bp, synthetic construct
  misc_feature 1..41: note = Description of sequence: crRNA, RNP ID: RNP16, Target Site ID: RefSeq16
  source 1..41: mol_type = other DNA
taatttctactcttgtagatgagtggtgaaaccagaacaat

SEQ ID NO: 98 - DNA, 41 bp, synthetic construct
  misc_feature 1..41: note = Description of sequence: crRNA, RNP ID: RNP17, Target Site ID: RefSeq17
  source 1..41: mol_type = other DNA
taatttctactcttgtagatgttgacatagaagaggcacaa

SEQ ID NO: 99 - DNA, 41 bp, synthetic construct
  misc_feature 1..41: note = Description of sequence: crRNA, RNP ID: RNP18, Target Site ID: RefSeq18
  source 1..41: mol_type = other DNA
taatttctactcttgtagattgttattttaaggaaattcga

SEQ ID NO: 100 - DNA, 41 bp, synthetic construct
  misc_feature 1..41: note = Description of sequence: crRNA, RNP ID: RNP19, Target Site ID: RefSeq19
  source 1..41: mol_type = other DNA
taatttctactcttgtagatagaggaaagtcctgtacatcg

SEQ ID NO: 101 - DNA, 41 bp, synthetic construct
  misc_feature 1..41: note = Description of sequence: crRNA, RNP ID: RNP20, Target Site ID: RefSeq20
  source 1..41: mol_type = other DNA
taatttctactcttgtagatcctttcttgcagatggaaaaa

SEQ ID NO: 102 - DNA, 41 bp, synthetic construct
  misc_feature 1..41: note = Description of sequence: crRNA, RNP ID: RNP21, Target Site ID: RefSeq21
  source 1..41: mol_type = other DNA
taatttctactcttgtagatcactttttagagtggtgaaac

SEQ ID NO: 103 - DNA, 41 bp, synthetic construct
  misc_feature 1..41: note = Description of sequence: crRNA, RNP ID: RNP22, Target Site ID: RefSeq22
  source 1..41: mol_type = other DNA
taatttctactcttgtagataggaaattcgaaatgagtccc

SEQ ID NO: 104 - DNA, 41 bp, synthetic construct
  misc_feature 1..41: note = Description of sequence: crRNA, RNP ID: RNP23, Target Site ID: RefSeq23
  source 1..41: mol_type = other DNA
taatttctactcttgtagatgaggaaagtcctgtacatcgg

SEQ ID NO: 105 - DNA, 41 bp, synthetic construct
  misc_feature 1..41: note = Description of sequence: crRNA, RNP ID: RNP24, Target Site ID: RefSeq24
  source 1..41: mol_type = other DNA
taatttctactcttgtagatgcctttcttgcagatggaaaa

SEQ ID NO: 106 - DNA, 41 bp, synthetic construct
  misc_feature 1..41: note = Description of sequence: crRNA, RNP ID: RNP25, Target Site ID: RefSeq25
  source 1..41: mol_type = other DNA
taatttctactcttgtagatgggcaagaaaggtcttacatt

SEQ ID NO: 107 - DNA, 41 bp, synthetic construct
  misc_feature 1..41: note = Description of sequence: crRNA, RNP ID: RNP26, Target Site ID: RefSeq26
  source 1..41: mol_type = other DNA
taatttctactcttgtagataaggaaattcgaaatgagtcc

SEQ ID NO: 108 - DNA, 41 bp, synthetic construct
  misc_feature 1..41: note = Description of sequence: crRNA, RNP ID: RNP27, Target Site ID: RefSeq27
  source 1..41: mol_type = other DNA
taatttctactcttgtagataaacctgtatgaagtttacct

SEQ ID NO: 109 - DNA, 41 bp, synthetic construct
  misc_feature 1..41: note = Description of sequence: crRNA, RNP ID: RNP28, Target Site ID: RefSeq28
  source 1..41: mol_type = other DNA
taatttctactcttgtagatagagtggtgaaaccagaacaa

SEQ ID NO: 110 - DNA, 41 bp, synthetic construct
  misc_feature 1..41: note = Description of sequence: crRNA, RNP ID: RNP29, Target Site ID: RefSeq29
  source 1..41: mol_type = other DNA
taatttctactcttgtagatagatgatcacagtcgtgttaa

SEQ ID NO: 111 - RNA, 20 nt, synthetic construct
  misc_feature 1..20: note = Description of sequence: AsCas12a stem loop (scaffold sequence)
  source 1..20: mol_type = other RNA
taatttctactcttgtagat

SEQ ID NO: 112 - DNA, 66 bp, synthetic construct
  misc_feature 1..66: note = Description of sequence: gRNA with DNA extension
  source 1..66: mol_type = other DNA
  misc_feature 1..25: note = DNA sequence, deoxyribose scaffold
  misc_feature 26..66: note = DNA sequence, deoxyribose scaffold
  misc_feature 26: note = t is uracil
  misc_feature 29: note = t is uracil
  misc_feature 30: note = t is uracil
  misc_feature 31: note = t is uracil
  misc_feature 33: note = t is uracil
  misc_feature 36: note = t is uracil
  misc_feature 38: note = t is uracil
  misc_feature 39: note = t is uracil
  misc_feature 41: note = t is uracil
  misc_feature 45: note = t is uracil
  misc_feature 49: note = t is uracil
  misc_feature 52: note = t is uracil
  misc_feature 66: note = t is uracil
atgtgtttttgtcaaaagacctttttaatttctactcttgtagatgagtggtgaaaccagaacaat

SEQ ID NO: 113 - protein, 1334 aa, synthetic construct
  REGION 1..1334: note = Description of sequence: His-AsCpfl-sNLS-sNLS H800A amino acid sequence
MGHHHHHHGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 114 - protein, 1363 aa, synthetic construct
  REGION 1..1363: note = Description of sequence: Cpfl variant 1 amino acid
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 115 - protein, 1363 aa, synthetic construct
  REGION 1..1363: note = Description of sequence: Cpfl variant 2 amino acid sequence
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 116 - protein, 1363 aa, synthetic construct
  REGION 1..1363: note = Description of sequence: Cpfl variant 3 amino acid sequence
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 117 - protein, 1331 aa, synthetic construct
  REGION 1..1331: note = Description of sequence: Cpfl variant 4 amino acid sequence
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 118 - protein, 1331 aa, synthetic construct
  REGION 1..1331: note = Description of sequence: Cpfl variant 5 amino acid sequence
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 119 - protein, 1363 aa, synthetic construct; identical to SEQ ID NO: 114
  REGION 1..1363: note = Description of sequence: Cpfl variant 6 amino acid sequence

SEQ ID NO: 120 - protein, 1361 aa, synthetic construct
  REGION 1..1361: note = Description of sequence: Cpfl variant 7 amino acid sequence
MGRDPGKPIPNPLLGLDSTAPKKKRKVGIHGVPAATQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNPKKKRKVKLAAALEHHHHHH

SEQ ID NO: 121 - protein, 1307 aa, synthetic construct
  REGION 1..1307: note = Description of sequence: Exemplary AsCpfl wild-type amino acid sequence
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 122 - DNA, 65 bp, synthetic construct
  misc_feature 1..65: note = Description of sequence: Forward Primer
  source 1..65: mol_type = other DNA
acactctttccctacacgacgctcttccgatctctcactttaaacaagaaattgagaaatgaagc

SEQ ID NO: 123 - DNA, 59 bp, synthetic construct
  misc_feature 1..59: note = Description of sequence: Reverse Primer
  source 1..59: mol_type = other DNA
gtgactggagttcagacgtgtgctcttccgatctggtaacagattagtagctgagaacc

SEQ ID NO: 124 - DNA, 592 bp, synthetic construct
  misc_feature 1..592: note = Description of sequence: Amplicon
  source 1..592: mol_type = other DNA
acactctttccctacacgacgctcttccgatctctcactttaaacaagaaattgagaaatgaagctggtgattcagggactccaaaatctggccaggtagtataatgaaagtgagatattgttctggtttcaccactctaaaaagtgaaaatcaagggagaagttagttctaacttttttcttttaaaaaactacttcagaaagatcatgtgttttatataattctttaaagccatataggctgtttatactatgatttttccatttgttttctgacaatcccatatacaaactagggaagattttctttagtggaaatttgtgatgtattaataatagctaacgcatacaacacttactattttggaggcctgttctaagcactttacttattaactcaatccacacaataatcctattaaagcccattttcctgatgaggaaactgaaacaaagggtaagatagtttgcccaaggtcacacagctggaacatggcagaaccaggatttgaagcaacctggggcagcctttgggttctcagctactaatctgttaccagatcggaagagcacacgtctgaactccagtcac

SEQ ID NO: 125 - DNA, 34 bp, synthetic construct
  misc_feature 1..34: note = Description of sequence: dsODN tag (top)
  source 1..34: mol_type = other DNA
  modified_base 1^2: mod_base = OTHER; note = A phosphorothioate llinkage is present between these bases
  modified_base 2^3: mod_base = OTHER; note = A phosphorothioate llinkage is present between these bases
  modified_base 32^33: mod_base = OTHER; note = A phosphorothioate llinkage is present between these bases
  modified_base 33^34: mod_base = OTHER; note = A phosphorothioate llinkage is present between these bases
  modified_base 1: mod_base = OTHER; note = 5' phosphorylation
gtttaattgagttgtcatatgttaataacggtat

SEQ ID NO: 126 - DNA, 34 bp, synthetic construct
  misc_feature 1..34: note = Description of sequence: dsODN tag (bottom)
  source 1..34: mol_type = other DNA
  modified_base 1^2: mod_base = OTHER; note = A phosphorothioate llinkage is present between these bases
  modified_base 2^3: mod_base = OTHER; note = A phosphorothioate llinkage is present between these bases
  modified_base 32^33: mod_base = OTHER; note = A phosphorothioate llinkage is present between these bases
  modified_base 33^34: mod_base = OTHER; note = A phosphorothioate llinkage is present between these bases
  modified_base 1: mod_base = OTHER; note = 5' phosphorylation
ataccgttattaacatatgacaactcaattaaac

SEQ ID NO: 127 - DNA, 29 bp, synthetic construct
  misc_feature 1..29: note = Description of sequence: P5 primer
  source 1..29: mol_type = other DNA
aatgatacggcgaccaccgagatctacac

SEQ ID NO: 128 - DNA, 68 bp, synthetic construct
  misc_feature 1..68: note = Description of sequence: dsODN tag specific primer (minus)
  source 1..68: mol_type = other DNA
gtgactggagttcagacgtgtgctcttccgatctataccgttattaacatatgacaactcaattaaac

SEQ ID NO: 129 - DNA, 68 bp, synthetic construct
  misc_feature 1..68: note = Description of sequence: dsODN tag specific primer (plus)
  source 1..68: mol_type = other DNA
gtgactggagttcagacgtgtgctcttccgatctgtttaattgagttgtcatatgttaataacggtat

SEQ ID NO: 130 - protein, 1307 aa, synthetic construct
  REGION 1..1307: note = Description of sequence: Cas12a variant 4 amino acid sequence
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 131 - DNA, 250 bp, synthetic construct
  misc_feature 1..250: note = Description of sequence: target sequence of PTPN2 gene
  source 1..250: mol_type = other DNA
ccttgattttcactttttagagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttggtaagtgcttcttacgatct